SEQUENCE LISTING (71 sequences)

SEQ ID NO: 1 - DNA, 22 bp, Homo sapiens
ccacacacttccttacattcca

SEQ ID NO: 2 - DNA, 22 bp, Artificial Sequence
  Description of Artificial Sequence Synthetic isolated miR-206 TS with substitution at position 2 oligonucleotide
ccacacacttccttacattcga

SEQ ID NO: 3 - DNA, 22 bp, Artificial Sequence
  Description of Artificial Sequence Synthetic isolated miR-206 TS with substitution at position 3 oligonucleotide
ccacacacttccttacattgca

SEQ ID NO: 4 - DNA, 22 bp, Artificial Sequence
  Description of Artificial Sequence Synthetic isolated miR-206 TS with substitution at position 4 oligonucleotide
ccacacacttccttacatacca

SEQ ID NO: 5 - DNA, 22 bp, Artificial Sequence
  Description of Artificial Sequence Synthetic isolated miR-206 TS with substitution at position 5 oligonucleotide
ccacacacttccttacaatcca

SEQ ID NO: 6 - DNA, 22 bp, Artificial Sequence
  Description of Artificial Sequence Synthetic isolated miR-206 TS with substitution at position 6 oligonucleotide
ccacacacttccttactttcca

SEQ ID NO: 7 - DNA, 22 bp, Artificial Sequence
  Description of Artificial Sequence Synthetic isolated miR-206 TS with substitution at position 7 oligonucleotide
ccacacacttccttagattcca

SEQ ID NO: 8 - DNA, 22 bp, Artificial Sequence
  Description of Artificial Sequence Synthetic isolated miR-206 TS with substitution at position 8 oligonucleotide
ccacacacttcctttcattcca

SEQ ID NO: 9 - DNA, 22 bp, Artificial Sequence
  Description of Artificial Sequence Synthetic isolated miR-206 TS with substitution at position 12 oligonucleotide
ccacacacttgcttacattcca

SEQ ID NO: 10 - DNA, 22 bp, Artificial Sequence
  Description of Artificial Sequence Synthetic isolated miR-206 TS with substitution at position 13 oligonucleotide
ccacacactaccttacattcca

SEQ ID NO: 11 - DNA, 22 bp, Artificial Sequence
  Description of Artificial Sequence Synthetic isolated miR-206 TS with substitution at position 14 oligonucleotide
ccacacacatccttacattcca

SEQ ID NO: 12 - DNA, 22 bp, Artificial Sequence
  Description of Artificial Sequence Synthetic isolated miR-206 TS with substitution at position 15 oligonucleotide
ccacacagttccttacattcca

SEQ ID NO: 13 - DNA, 22 bp, Artificial Sequence
  Description of Artificial Sequence Synthetic isolated miR-206 TS with substitution at position 16 oligonucleotide
ccacactcttccttacattcca

SEQ ID NO: 14 - DNA, 22 bp, Artificial Sequence
  Description of Artificial Sequence Synthetic isolated miR-206 TS with substitution at position 3 oligonucleotide
ccacacacttccttacattaca

SEQ ID NO: 15 - DNA, 22 bp, Artificial Sequence
  Description of Artificial Sequence Synthetic isolated miR-206 TS with substitution at position 3 oligonucleotide
ccacacacttccttacatttca

SEQ ID NO: 16 - RNA, 22 nt, Homo sapiens
uggaauguaaggaagugugugg

SEQ ID NO: 17 - RNA, 21 nt, Homo sapiens
uggaauguaaagaaguaugua

SEQ ID NO: 18 - DNA, 22 bp, Homo sapiens
aaagctagaaagtcaagtggaa

SEQ ID NO: 19 - DNA, 21 bp, Homo sapiens
tacatacttctttacattcca

SEQ ID NO: 20 - DNA, 22 bp, Artificial Sequence
  Description of Artificial Sequence Synthetic isolated miR-206 TS with substitution at position 9 oligonucleotide
ccacacacttcctaacattcca

SEQ ID NO: 21 - DNA, 22 bp, Artificial Sequence
  Description of Artificial Sequence Synthetic isolated miR-206 TS with substitution at position 10 oligonucleotide
ccacacacttccatacattcca

SEQ ID NO: 22 - DNA, 22 bp, Artificial Sequence
  Description of Artificial Sequence Synthetic isolated miR-206 TS with substitution at position 12 oligonucleotide
ccacacacttacttacattcca

SEQ ID NO: 23 - DNA, 28 bp, Artificial Sequence
  Description of Artificial Sequence Synthetic primer
ctagtatacatacttctttacattccat

SEQ ID NO: 24 - DNA, 28 bp, Artificial Sequence
  Description of Artificial Sequence Synthetic primer
ctagatggaatgtaaagaagtatgtata

SEQ ID NO: 25 - DNA, 28 bp, Artificial Sequence
  Description of Artificial Sequence Synthetic primer
ctaggccacacacttccttacattccat

SEQ ID NO: 26 - DNA, 28 bp, Artificial Sequence
  Description of Artificial Sequence Synthetic primer
ctagatggaatgtaaggaagtgtgtggc

SEQ ID NO: 27 - DNA, 25 bp, Artificial Sequence
  Description of Artificial Sequence Synthetic primer
cgaaagctagaaagtcaagtggaat

SEQ ID NO: 28 - DNA, 29 bp, Artificial Sequence
  Description of Artificial Sequence Synthetic primer
ctagattccacttgactttctagctttcg

SEQ ID NO: 29 - DNA, 26 bp, Artificial Sequence
  Description of Artificial Sequence Synthetic primer
cctccacacacttccttacattccat

SEQ ID NO: 30 - DNA, 30 bp, Artificial Sequence
  Description of Artificial Sequence Synthetic primer
ctagatggaatgtaaggaagtgtgtggagg

SEQ ID NO: 31 - DNA, 28 bp, Artificial Sequence
  Description of Artificial Sequence Synthetic primer
ctaggccacacacttccttacattccat

SEQ ID NO: 32 - DNA, 28 bp, Artificial Sequence
  Description of Artificial Sequence Synthetic primer
ctagatggaatgtaaggaagtgtgtggc

SEQ ID NO: 33 - DNA, 90 bp, Artificial Sequence
  Description of Artificial Sequence Synthetic DNA containing 3 repeats of mutated miR-206TS oligonucleotide
aggcctccacacacttccttacattgcatctaggccacacacttccttacattgcatctaggccacacacttccttacattgcatctaga

SEQ ID NO: 34 - DNA, 22 bp, Artificial Sequence
  Description of Artificial Sequence Synthetic primer
ccagtgtacatcgcgaacaaac

SEQ ID NO: 35 - DNA, 26 bp, Artificial Sequence
  Description of Artificial Sequence Synthetic primer
ataatgtacaggccgccccgactcta

SEQ ID NO: 36 - DNA, 37 bp, Artificial Sequence
  Description of Artificial Sequence Synthetic primer
aaagacgatgacgacaagggctctgagctggagacgg

SEQ ID NO: 37 - DNA, 27 bp, Artificial Sequence
  Description of Artificial Sequence Synthetic primer
aagttcgcgagtcaactgttctcccag

SEQ ID NO: 38 - DNA, 37 bp, Artificial Sequence
  Description of Artificial Sequence Synthetic primer
aatcggatccatggactacaaagacgatgacgacaag

SEQ ID NO: 39 - DNA, 27 bp, Artificial Sequence
  Description of Artificial Sequence Synthetic primer
aagttcgcgagtcaactgttctcccag

SEQ ID NO: 40 - DNA, 27 bp, Artificial Sequence
  Description of Artificial Sequence Synthetic primer
aagttctagagtcaactgttctcccag

SEQ ID NO: 41 - DNA, 90 bp, Artificial Sequence; identical to SEQ ID NO: 33
  Description of Artificial Sequence Synthetic DNA containing 3 repeats of mutated miR-206TS oligonucleotide

SEQ ID NO: 42 - DNA, 19 bp, Artificial Sequence
  Description of Artificial Sequence Synthetic primer
gggctctgagctggagacg

SEQ ID NO: 43 - DNA, 20 bp, Artificial Sequence
  Description of Artificial Sequence Synthetic primer
caccttgtccacagcatcca

SEQ ID NO: 44 - DNA, 20 bp, Artificial Sequence
  Description of Artificial Sequence Synthetic primer
ccactcgggcaaagaggggg

SEQ ID NO: 45 - DNA, 21 bp, Artificial Sequence
  Description of Artificial Sequence Synthetic primer
cccaagctttattgcggtagt

SEQ ID NO: 46 - DNA, 24 bp, Artificial Sequence
  Description of Artificial Sequence Synthetic primer
ggctagcctatagtgagtcgtatt

SEQ ID NO: 47 - DNA, 20 bp, Artificial Sequence
  Description of Artificial Sequence Synthetic primer
ttcgctaagagcaccctgat

SEQ ID NO: 48 - DNA, 20 bp, Artificial Sequence
  Description of Artificial Sequence Synthetic primer
ctcgtcccagtaggcaatgt

SEQ ID NO: 49 - DNA, 20 bp, Artificial Sequence
  Description of Artificial Sequence Synthetic primer
ccctgatcaagagcgaagag

SEQ ID NO: 50 - DNA, 20 bp, Artificial Sequence
  Description of Artificial Sequence Synthetic primer
catttcatctggagcgtcct

SEQ ID NO: 51 - DNA, 36 bp, Artificial Sequence
  Description of Artificial Sequence Synthetic oligonucleotide
ctcagaaaagctagaaagtcaagtggaagtttaaac

SEQ ID NO: 52 - DNA, 36 bp, Artificial Sequence
  Description of Artificial Sequence Synthetic oligonucleotide
ctcagaatacatacttctttacattccagtttaaac

SEQ ID NO: 53 - DNA, 36 bp, Artificial Sequence
  Description of Artificial Sequence Synthetic oligonucleotide
ctcagaccacacacttccttacattccagtttaaac

SEQ ID NO: 54 - DNA, 36 bp, Artificial Sequence
  Description of Artificial Sequence Synthetic oligonucleotide
ctcagaccacacacttccttacattgcagtttaaac

SEQ ID NO: 55 - DNA, 36 bp, Artificial Sequence
  Description of Artificial Sequence Synthetic oligonucleotide
ctcagaccacacacttcctaacattccagtttaaac

SEQ ID NO: 56 - DNA, 36 bp, Artificial Sequence
  Description of Artificial Sequence Synthetic oligonucleotide
ctcagaccacacacttccatacattccagtttaaac

SEQ ID NO: 57 - DNA, 36 bp, Artificial Sequence
  Description of Artificial Sequence Synthetic oligonucleotide
ctcagaccacacacttacttacattccagtttaaac

SEQ ID NO: 58 - DNA, 36 bp, Artificial Sequence
  Description of Artificial Sequence Synthetic oligonucleotide
ctcagaccacacactaccttacattccagtttaaac

SEQ ID NO: 59 - DNA, 36 bp, Artificial Sequence
  Description of Artificial Sequence Synthetic oligonucleotide
ctcagaccacacacatccttacattccagtttaaac

SEQ ID NO: 60 - DNA, 36 bp, Artificial Sequence
  Description of Artificial Sequence Synthetic oligonucleotide
ctcagaccacacagttccttacattccagtttaaac

SEQ ID NO: 61 - DNA, 36 bp, Artificial Sequence
  Description of Artificial Sequence Synthetic oligonucleotide
ctcagaccacactcttccttacattccagtttaaac

SEQ ID NO: 62 - DNA, 36 bp, Artificial Sequence
  Description of Artificial Sequence Synthetic oligonucleotide
ctcagaccacacacttccttacattcgagtttaaac

SEQ ID NO: 63 - DNA, 36 bp, Artificial Sequence
  Description of Artificial Sequence Synthetic oligonucleotide
ctcagaccacacacttccttacataccagtttaaac

SEQ ID NO: 64 - DNA, 36 bp, Artificial Sequence
  Description of Artificial Sequence Synthetic oligonucleotide
ctcagaccacacacttccttacaatccagtttaaac

SEQ ID NO: 65 - DNA, 36 bp, Artificial Sequence
  Description of Artificial Sequence Synthetic oligonucleotide
ctcagaccacacacttccttactttccagtttaaac

SEQ ID NO: 66 - DNA, 36 bp, Artificial Sequence
  Description of Artificial Sequence Synthetic oligonucleotide
ctcagaccacacacttccttagattccagtttaaac

SEQ ID NO: 67 - DNA, 36 bp, Artificial Sequence
  Description of Artificial Sequence Synthetic oligonucleotide
ctcagaccacacacttcctttcattccagtttaaac

SEQ ID NO: 68 - DNA, 36 bp, Artificial Sequence
  Description of Artificial Sequence Synthetic oligonucleotide
ctcagaccacacacttccttacattacagtttaaac

SEQ ID NO: 69 - DNA, 36 bp, Artificial Sequence
  Description of Artificial Sequence Synthetic oligonucleotide
ctcagaccacacacttccttacatttcagtttaaac

SEQ ID NO: 70 - DNA, 36 bp, Artificial Sequence; identical to SEQ ID NO: 54
  Description of Artificial Sequence Synthetic oligonucleotide

SEQ ID NO: 71 - DNA, 16 bp, Artificial Sequence
  Description of Artificial Sequence Synthetic primer
tttttttttttttttt

(19) United States
(12) Patent Application Publication
(10) Pub. No.: US 2013/0281508 A1
(43) Pub. Date: Oct. 24, 2013

(54) MicroRNA target site for cell- or tissue-specific inhibition of expression of a transgene

(71) Applicants: Henry Fechner, Luckau (DE); Geisler Anja, Berlin (DE)

(72) Inventors: Henry Fechner, Luckau (DE); Geisler Anja, Berlin (DE)

(73) Assignees: Charite-Universitatsmedizin Berlin, Berlin (DE); Technische Universitat Berlin, Berlin (DE)

(21) Appl. No.: 13/450,392

(22) Filed: Apr. 18, 2012

(51) Int. Cl. A61K 31/7088; A61P 9/12; A61P 9/00; C12N 15/113; C12N 15/63

(52) U.S. Cl. 514/44 A; 536/24.5; 435/320.1; 514/44 R

(57) ABSTRACT

The present invention is directed to an isolated miR-206 target site, comprising or consisting of a nucleic acid sequence with a sequence identity of at least 80% compared to wild type miR-206 target site with SEQ ID No. 1, characterized in that the nucleic acid sequence comprises at least one nucleotide substitution at a position from nucleotide 2 to 8 and/or at least one nucleotide substitution at a position from nucleotide 12 to 16 of SEQ D No. 1, wherein the nucleotide positions of SEQ ID No. 1 are numbered from the 3′- to the 5′-end; as well as to an expression cassette, vector and pharmaceutical composition comprising at least one isolated miR-206 target site of the invention.

### FIELD OF THE INVENTION

The present invention is directed to novel microRNA target sites which allow for cell- or tissue specific modulation of expression of a transgene.

### BACKGROUND OF THE INVENTION

Heart diseases are one of the most important causes of death worldwide. Despite development of novel therapeutic modalities in the last decades, options for treatment of many heart diseases are still limited. Gene transfer to the myocardium has become a promising therapeutic strategy, as it allows highly specific modulation of singular genes or gene networks. Its suitability for employment in heart diseases has been demonstrated in several animal models like e.g. models of myocardial ischemia, heart failure and genetic disorders. Recently, clinical trials for treatment of patients with advanced heart failure have been initiated.

As cardiac gene transfer efficiency of plasmid DNA is low even after local injection, viral vector systems have gained increasing interest. Among these, the adeno-associated virus (AAV) vectors are currently the most potent and promising vectors used for delivery of transgenes to the heart. AAV vectors have several advantages over other viral vector systems as they are not associated with any disease in humans. Furthermore, they allow long-term gene transfer in humans. Improvements in vector development resulted in so-called self-complementary (sc) AAV vectors harbouring double stranded genomes. These vectors allow an extremely rapid and efficient expression of transgenes enabling even treatment of acute virus infections of the heart in a murine model. Most importantly, identification of novel AAV serotypes resulted in the development of AAV vectors suitable for an efficient cardiac gene transfer upon systemic application in mice as shown for AAV9. However, AAV9 vectors exhibit a broad tissue tropism and allow also transduction of the liver upon intravascular administration. Reduction of AAV-mediated transgene expression in non-target organs and tissues appears to be a desirable aim to reduce or avoid unwanted side effects in gene therapy. Transcriptional control of gene expression is a promising approach to overcome this limitation. The use of tissue or cell type specific promoters can lead to an efficient and predominant gene expression in the target organ, tissue or cell type. However, such an approach may not completely prevent transgene expression in non-target organs, tissues or cells.

Alternatively or in addition to the use of specific promoters, it has been found that expression of the transgene can also be regulated by use of microRNAs (miRs) and its specific target sites. MiRs are a group of endogenous, short and non-coding RNA molecules that have a central role as key post-transcriptional regulators of gene activity. These molecules are transcribed by polymerase II as long hairpin precursor transcripts. After sequential processing steps, a double stranded 18-24 nucleotide long miR is incorporated into the RNA-induced silencing complex. Only one strand, the guide strand of the miR duplex, remains stably associated with RNA-induced silencing complex and forms the mature miR, whereas the opposite strand is disposed. By pairing with partially complementary sites, the target sites of a given miR, in the 3′-untranslated regions (3′-UTRs) mature miRs mediate post-transcriptional silencing of genes.

Previous studies have demonstrated that insertion of specific miR target sites into the 3′-UTR of a gene expression cassette reduces expression levels of the transgene in cells and organs with high levels of corresponding miR expression, see e.g. US 2010/0186103 A1, WO 2010/055413 A1, WO 2007/070483 and Geisler et al. (Gene Therapy (2011) 18, 199-209). As reported by Geisler et al., introduction of miR-122 target site into the 3′-UTR of an AAV9 vector construct led to silencing of transgene expression in the liver, whereas expression of the transgene in the heart was maintained.

It is an aim of the present invention to provide novel means and strategies to improve cell or tissue specific expression of transgenes and, thereby, to extend the options for gene therapy.

### DETAILED DESCRIPTION

According to an aspect of the invention, novel isolated miR-206 target sites are provided wherein the isolated mi-R-206 target site of the invention exhibits at least one mutation compared to wild type miR-206 target site.

The present invention is based on the following unexpected findings.

Although the general concept of modulating transgene expression by use of specific miR target sites appears to be promising, currently there is no strategy available to reduce transgene expression in skeletal muscle while expression level in the heart is maintained. With miR-206 there is a miR species available which is highly expressed in skeletal muscle and which is virtually absent in the heart. However, experiments revealed that introduction of miR-206 target site into the 3′-UTR did not yield the expected result. Use of a cardiotropic AAV9 vector with a miR-206 target site in the 3′-UTR of the gene to be expressed in deed exhibited reduced expression of the transgene in skeletal muscle. However, expression of the transgene was also silenced in the heart. This effect appears to be due to the high homology between miR-206 and miR-1. Thus, miR-1 is capable of binding efficiently to miR-206 target site and thereby to silence transgene expression. Since miR-1 is highly expressed in the heart, the use of wild type miR-206 target site in the vector construct prohibits efficient transgene expression in both tissues, the heart and skeletal muscle.

It has now surprisingly been found that introduction of a mutation, e.g. of a nucleotide substitution, into the nucleic acid sequence of wild type miR-206 target site with SEQ ID No. 1 allows the generation of an isolated miR-206 target site of the invention which allows repression of expression of the transgene in cells and tissues with high expression of miR-206, whereas expression level of the transgene in cells and tissues with high expression of miR-1 is maintained.

Introduction of a nucleotide substitution at a position from nucleotide 12 to 16 of wild type miR-206 target site (wherein the nucleotide positions of wild type miR-206 are numbered from the 3′- to the 5′-end) yields an isolated miR-206 target site of the invention with improved binding efficiency to miR-206 wherein binding efficiency to miR-1 remains unaltered. Thus, the use of such an isolated miR-206 target site improves the difference in expression level of a transgene between cells or tissues expressing miR-206 and cells and tissues expressing miR-1 (and which substantially lack expression of miR-206).

Introduction of a mutation at a position from 2 to 7 of wild type miR-206 target site (wherein the nucleotide positions of wild type miR-206 are numbered from the 3′- to the 5′-end) yields an isolated miR-206 target site of the invention which exhibits maintained binding to miR-206 and lacks efficient binding of miR-1. Thus, the use of such an isolated miR-206 target site allows for efficient repression of transgene expression in cells and tissues with high levels of miR206, whereas transgene expression in cells and tissues that lack miR-206 expression but exhibit high level of miR-1 remains non-repressed.

Introduction of a nucleotide substitution at a nucleotide position 4, 6, 7, 8 and from nucleotide position 12 to 16 of wild type miR-206 target site (wherein the nucleotide positions of wild type miR-206 are numbered from the 3′- to the 5′-end) yields an isolated miR-206 target site of the invention with improved binding efficiency to miR-206. Thus, these mutations of miR-target site enhance inhibition of transgene expression levels compared to wild-type miR target site.

Unless defined otherwise, all technical and scientific terms used herein have the same meaning as commonly understood by one of ordinary skill in the art to which the invention pertains. In describing and claiming the present invention, the following terminology will be used in accordance with the definitions set out below.

As used in this specification and the appended claims, the singular forms “a”, “an” and “the” include singular and plural referents unless the content clearly dictates otherwise. Thus, for example, reference to “a nucleotide” includes one nucleotide and a combination of two or more nucleotides, whereas reference to “one nucleotide” effectively limits the meaning to only one nucleotide, thus, a combination of two or more nucleotides is excluded.

The present invention is directed to a miR-206 target site. A nucleic acid is a miR-206 target site if the nucleic acid comprises a nucleic acid sequence which allows binding of miR-206 and is capable of mediating miR-206 induced silencing of expression of a reporter gene if placed in the 3′-UTR of the reporter gene. Suitable assays for testing a given nucleic acid to be a miR-206 target site are well known in the art and a particularly useful assay is described in the experimental section below.

The present invention is directed to an isolated miR-206 target site, comprising or consisting of a nucleic acid sequence with a sequence identity of at least 80%, preferably of at least 90%, more preferably of at least 95%, compared to wild type miR-206 target site with SEQ ID No. 1, characterized in that the nucleic acid sequence comprises at least one nucleotide substitution at a position from nucleotide 2 to 8, preferably 2 to 7, and/or at least one nucleotide substitution at a position from nucleotide 12 to 16 of SEQ D No. 1, wherein the nucleotide positions of SEQ ID No. 1 are numbered from the 3′- to the 5′-end.

The term “nucleic acid” is generally used in its art—recognized meaning to refer to a ribose nucleic acid (RNA) or deoxyribose nucleic acid (DNA) polymer, or analogue thereof, e.g., a nucleotide polymer comprising modifications of the nucleotides, a peptide nucleic acid, or the like. In certain applications, the nucleic acid can be a polymer that includes multiple monomer types, e.g., both RNA and DNA subunits. A nucleic acid can be e.g. a chromosome or chromosomal segment, a vector (e.g., an expression vector), an expression cassette, a naked DNA or RNA polymer, the product of a polymerase chain reaction (PCR), an oligonucleotide, a probe, etc. A nucleic acid can be e.g. single-stranded and/or fully or partially double-stranded. Unless otherwise indicated, a particular nucleic acid sequence of the invention comprises its corresponding complementary sequence, in addition to any sequence explicitly indicated. The term “nucleic acid sequence” or “nucleotide sequence” refers to a contiguous sequence of nucleotides in a single nucleic acid or to a representation, e.g., a character string, thereof. That is, a “nucleic acid sequence” or “nucleotide sequence” is a polymer of nucleotides (i.e. a nucleic acid) or a character string representing a nucleotide polymer, depending on context. Thus, the terms “nucleic acid” and “nucleic acid sequence” are used herein interchangingly. From any specified nucleotide sequence, either the given nucleic acid or the complementary nucleotide sequence (e.g. the corresponding complementary nucleic acid) can be determined.

The miR-206 target site of the invention is an isolated miR-206 target site and, thus, does not extend to naturally occurring miR-206 target sites which are within their original biological naturally-occurring context. In the context of the present invention, the term “isolated” refers to a biological material, such as a nucleic acid or nucleic acid sequence, which is substantially free from one or more components that normally accompany or interact with it in its naturally occurring environment. The isolated material optionally comprises material not found with the material in its natural environment and/or optionally lacks components or material usually found in combination with the isolated material in its natural environment. For example, a miR-206 target site of the invention is isolated in the sense of the present invention if said miR-206 target site is no longer in its natural environment. Any miR-206 target site qualifies as isolated that has been produced by genetic, recombinant or biotechnological means. If the isolated miR-206 target site of the invention is placed into a genetic environment that differs from the environment where it naturally occurs, the miR-206 target site still qualifies as isolated in the sense of the present invention. Thus, the isolated miR-206 target site of the invention which is part of an amplicon, an expression cassette or a vector always qualifies as being isolated in the sense of the invention.

The isolated miR-206 target site of the invention comprises or consists of a nucleic acid sequence with a sequence identity of at least 80%, preferably of at least 90%, more preferably of at least 95%, compared to wild type miR-206 target site with SEQ ID No. 1. Sequence identity is determined over the whole sequence length of the nucleic acid sequence with SEQ ID No. 1.

For the purpose of the present invention, sequence “identity” can objectively be determined by any of a number of methods. The skilled person is well aware of these methods and can choose a suitable method without undue burden. A variety of methods for determining relationships between two or more sequences (e.g. identity, similarity and/or homology) are available and well known in the art. The methods include manual alignment, computer assisted sequence alignment and combinations thereof, for example. A number of algorithms (which are generally computer implemented) for performing sequence alignment are widely available or can be produced by one of skill. The degree of identity between one nucleotide sequence and another can be determined by following the algorithm BLAST by Karlin and Altschul (Proc. Natl. Acad. Sci. USA 90: 5873-5877, 1993). Programs based on this algorithm (Altschul et al. (1990) J. Mol. Biol. 215: 403-410) may be used such as BLASTN. To analyze a nucleotide sequence according to BLASTN based on BLAST, the parameters are set, for example, as score=100 and word length=12. Default parameters of each program are used when using BLAST and Gapped BLAST program. Specific techniques for such analysis are known in the art (see http://www.ncbi.nim.nih.gov.).

The isolated miR-206 target site of the invention, comprising or consisting of a nucleic acid sequence which comprises at least one nucleotide substitution at a position from nucleotide 2 to 8, preferably 2 to 7, and/or at least one nucleotide substitution at a position from nucleotide 12 to 16 of SEQ D No. 1, wherein the nucleotide positions of SEQ ID No. 1 are numbered from the 3′- to the 5′-end.

Wild type miR-206 targeting site with SEQ ID No. 1 has the following nucleotide sequence and numbering:

wild type miR-206 TS with SEQ ID No. 1 (5′→3′):

| | |
|---|---|
| Nucleotide | |
| position: | 20 15 10 5 1 |
| Sequence: | 5′- cca cac act tcc tta cat tcc a -3′ |

Within the isolated miR-206 target site of the invention the nucleotide substitution is a transition (purin to purin or pyrimidin to pyrimidin base) or a transversion (purin to pyrimidin base or vice versa). Preferably the nucleotide substitution is a transition with an A for a T, a T for an A, a G for a C, a C for a G.

The nucleic acid sequence of the isolated miR-206 target site of the invention may comprise more than one nucleotide substitution, wherein one, more than one or all nucleotide substitutions are at a position selected from nucleotide 2, 3, 4, 5, 6, 7, 8, 12, 13, 14, 15, 16 of SEQ ID No. 1, wherein the nucleotide positions of SEQ ID No. 1 are numbered from the 3′- to the 5′-end.

The nucleic acid sequence of the isolated miR-206 target site of the invention may comprise more than one nucleotide substitution, wherein one, more than one or all nucleotide substitutions are at a position selected from nucleotide 2, 3, 4, 5, 6, 7, 12, 13, 14, 15, 16 of SEQ ID No. 1, wherein the nucleotide positions of SEQ ID No. 1 are numbered from the 3′- to the 5′-end.

The isolated miR-206 target site of the invention may comprise or consist of a nucleic acid sequence with SEQ ID No. 1, wherein the nucleic acid sequence comprises one nucleotide substitution at a position selected from nucleotide 2, 3, 4, 5, 6, 7, 8, 12, 13, 14, 15, 16 of SEQ ID No. 1, wherein the nucleotide positions of SEQ ID No. 1 are numbered from the 3′- to the 5′-end.

The isolated miR-206 target site of the invention may comprise or consist of a nucleic acid sequence with SEQ ID No. 1, wherein the nucleic acid sequence comprises one nucleotide substitution at a position selected from nucleotide 2, 3, 4, 5, 6, 7, 12, 13, 14, 15, 16 of SEQ ID No. 1, wherein the nucleotide positions of SEQ ID No. 1 are numbered from the 3′- to the 5′-end.

Preferably the isolated miR-206 target site of the invention comprises or consists of a nucleic acid sequence which comprises at least one nucleotide substitution at a position from nucleotide 2 to 8, preferably 2 to 7, of wild type miR-206 target site of SEQ D No. 1, wherein the nucleotide positions of SEQ ID No. 1 are numbered from the 3′- to the 5′-end. It has surprisingly been found that if a nucleotide substitution is introduced in this region, the resulting isolated miR-206 target site leads to strong suppression of transgene expression by miR-206 and virtually no suppression of transgene expression by miR-1. Thus, isolated miR-206 target sites of the invention with a nucleotide substitution in this region are particularly suitable to exclude unwanted expression of the transgene in skeletal muscle whereas the desired expression in the heart is not compromised.

In a preferred embodiment, the isolated miR-206 target site of the invention comprises or consists of a nucleic acid sequence of:

isolated miR-206 TS with SEQ ID No. 2 (5′→3′; nucleotide substitution given in bold):

| | |
|---|---|
| Nucleotide | |
| position: | 20 15 10 5 1 |
| Sequence: | 5′-cca cac act tcc tta cat tcg a-3′ |

isolated miR-206 TS with SEQ ID No. 3 (5′→3′; nucleotide substitution given in bold):

| | |
|---|---|
| Nucleotide | |
| position: | 20 15 10 5 1 |
| Sequence: | 5′-cca cac act tcc tta cat tgc a-3′ |

isolated miR-206 TS with SEQ ID No. 4 (5′→3′; nucleotide substitution given in bold):

| | |
|---|---|
| Nucleotide | |
| position: | 20 15 10 5 1 |
| Sequence: | 5′-cca cac act tcc tta cat acc a-3′ |

isolated miR-206 TS with SEQ ID No. 5 (5′→3′; nucleotide substitution given in bold):

| | |
|---|---|
| Nucleotide | |
| position: | 20 15 10 5 1 |
| Sequence: | 5′-cca cac act tcc tta caa tcc a-3′ |

isolated miR-206 TS with SEQ ID No. 6 (5′→3′; nucleotide substitution given in bold):

| | |
|---|---|
| Nucleotide | |
| position: | 20 15 10 5 1 |
| Sequence: | 5′-cca cac act tcc tta ctt tcc a-3′ |

isolated miR-206 TS with SEQ ID No. 7 (5′→3′; nucleotide substitution given in bold):

| | |
|---|---|
| Nucleotide | |
| position: | 20 15 10 5 1 |
| Sequence: | 5′-cca cac act tcc tta gat tcc a-3′ |

isolated miR-206 TS with SEQ ID No. 8 (5′→3′; nucleotide substitution given in bold):

| | |
|---|---|
| Nucleotide | |
| position: | 20 15 10 5 1 |
| Sequence: | 5′-cca cac act tcc ttt cat tcc a-3′ |

isolated miR-206 TS with SEQ ID No. 9 (5′→3′; nucleotide substitution given in bold):

| | |
|---|---|
| Nucleotide | |
| position: | 20 15 10 5 1 |
| Sequence: | 5′-cca cac act tgc tta cat tcc a-3′ |

isolated miR-206 TS with SEQ ID No. 10 (5′→3′; nucleotide substitution given in bold):

| | |
|---|---|
| Nucleotide | |
| position: | 20 15 10 5 1 |
| Sequence: | 5′-cca cac act acc tta cat tcc a-3′ |

isolated miR-206 TS with SEQ ID No. 11 (5′→3′; nucleotide substitution given in bold):

| | |
|---|---|
| Nucleotide | |
| position: | 20 15 10 5 1 |
| Sequence: | 5′-cca cac aca tcc tta cat tcc a-3′ |

isolated miR-206 TS with SEQ ID No. 12 (5′→3′; nucleotide substitution given in bold):

| | |
|---|---|
| Nucleotide | |
| position: | 20 15 10 5 1 |
| Sequence: | 5′-cca cac agt tcc tta cat tcc a-3′ |

isolated miR-206 TS with SEQ ID No. 13 (5′→3′; nucleotide substitution given in bold):

| | |
|---|---|
| Nucleotide | |
| position: | 20 15 10 5 1 |
| Sequence: | 5′-cca cac tct tcc tta cat tcc a-3′ |

isolated miR-206 TS with SEQ ID No. 14 (5′→3′; nucleotide substitution given in bold):

| | |
|---|---|
| Nucleotide | |
| position: | 20 15 10 5 1 |
| Sequence: | 5′-cca cac act tcc tta cat tac a-3′ |

isolated miR-206 TS with SEQ ID No. 15 (5′→3′; nucleotide substitution given in bold):

| | |
|---|---|
| Nucleotide | |
| position: | 20 15 10 5 1 |
| Sequence: | 5′-cca cac act tcc tta cat ttc a-3′ |

isolated miR-206 TS with SEQ ID No. 22 (5′→3′; nucleotide substitution given in bold):

| | |
|---|---|
| Nucleotide | |
| position: | 20 15 10 5 1 |
| Sequence: | 5′-cca cac act tac tta cat tcc a-3′ |

In a particularly preferred embodiment, the isolated miR-206 target site of the invention consists or comprises a nucleotide sequence with SEQ ID No. 2, SEQ ID No. 3, SEQ ID No. 4, SEQ ID No. 5, SEQ ID No. 6, SE ID No. 7, SEQ ID No. 8, SEQ ID No. 14 or SEQ ID No. 15.

In an even more preferred embodiment, the isolated miR-206 target site of the invention consists or comprises a nucleotide sequence with SEQ ID No. 2, SEQ ID No. 3, SEQ ID No. 4, SEQ ID No. 5, SEQ ID No. 6, SE ID No. 7, SEQ ID No. 14 or SEQ ID No. 15.

The present invention is also directed to an expression cassette comprising a promoter, a nucleic acid sequence to be expressed, and at least one isolated miR-206 target site of the invention. An expression cassette is a nucleic acid molecule comprising nucleic acid sequences that, when brought into the right context, are capable of mediating expression of a certain nucleic acid sequence of the expression cassette. Thus, an expression cassette comprises, beside the nucleic acid sequence to be expressed, further nucleic acid sequences which are capable of mediating, supporting and/or regulating expression of the nucleic acid sequence to be expressed. The expression cassette of the present invention may comprise more than one isolated miR-206 target sites of the invention. Typically the expression cassette comprises 1 to 10 copies of the isolated miR-206 target sequence of the invention.

In the expression cassette of the invention, the nucleic acid to be expressed may comprise an open reading frame encoding for a polypeptide. However, it is also possible that the nucleic acid to be expressed does not encode for a polypeptide but encodes for a functional nucleic acid like e.g. an antisense molecule or an aptamer.

In the expression cassette, the isolated miR-206 target site of the present invention is preferably located in a 3′-untranslated region (UTR) of the nucleic acid sequence to be expressed.

In another aspect, the present invention is directed to a vector comprising at least one isolated miR-206 target site of the invention or at least one expression cassette of the invention. A vector is a nucleic acid capable of being used as vehicle to transfer genetic material into a cell, tissue or organism. Typically a vector is a plasmid, a viral vector, a cosmid or an artificial chromosome. In a preferred embodiment the vector of the invention is a plasmid or a viral vector. Particularly preferred, the vector is an adeno-associated viral (AAV) vector, a recombinant AAV vector. The vector may be any of AAV 1, AAV 2, AAV 3, AAV 4, AAV 5, AAV 6, AAV 7, AAV 8, AAV 9, AAV 10, AAV 11, AAV 12 or any other known or novel serotype. In a particularly preferred embodiment the vector is AAV 1 or AAV 9.

The vector of the invention can comprise a nucleic acid sequence to be expressed operably linked to a promoter and at least one isolated miR-206 target site of the invention, wherein the isolated miR-206 target site of the invention is preferably located in a 3′-untranslated region (3′-UTR) of the nucleic acid sequence to be expressed.

In the vector of the invention, the nucleic acid to be expressed preferably comprises a nucleic acid sequence encoding for a polypeptide. However, it is also possible that the nucleic acid to be expressed does not encode for a polypeptide but encodes for a functional nucleic acid like e.g. an antisense molecule or an aptamer.

According to another aspect, the present invention is directed to a pharmaceutical composition comprising an isolated miR-206 target site of the invention, an expression cassette of the invention or a vector of the invention and at least one pharmaceutically acceptable excipient. The term “excipient” is used herein to describe any ingredient other than the compound of the invention. The choice of excipient will to a large extent depend on the particular mode of administration of the pharmaceutical composition.

According to a further aspect, the present invention is directed to a method of treatment of a disease comprising the step of administering a patient in need of such treatment an effective amount of a pharmaceutical composition of the invention.

As used herein, the term “treating” refers to reversing, alleviating or inhibiting the progress of a disease, disorder or condition, or one or more symptoms of such disease, disorder or condition, to which such term applies. As used herein, “treating” may also refer to decreasing the probability or incidence of the occurrence of a disease, disorder or condition in a mammal as compared to an untreated control population, or as compared to the same mammal prior to treatment. For example, as used herein, “treating” may refer to preventing a disease, disorder or condition, and may include delaying or preventing the onset of a disease, disorder or condition, or delaying or preventing the symptoms associated with a disease, disorder or condition. As used herein, “treating” may also refer to reducing the severity of a disease, disorder or condition or symptoms associated with such disease, disorder or condition prior to a mammal's affliction with the disease, disorder or condition. Such prevention or reduction of the severity of a disease, disorder or condition prior to affliction relates to the administration of the composition of the present invention, as described herein, to a subject that is not at the time of administration afflicted with the disease, disorder or condition. As used herein “treating” may also refer to preventing the recurrence of a disease, disorder or condition or of one or more symptoms associated with such disease, disorder or condition. The terms “treatment” and “therapeutically,” as used herein, refer to the act of treating, as “treating” is defined above.

According to the present invention, the pharmaceutical composition of the invention is administered at an effective amount. An “effective amount” is the amount of the pharmaceutical composition that upon administration to a patient yields a measurable therapeutic effect with regard to the disease of interest.

In the method of the invention the pharmaceutical composition is administered to a patient in order to treat a disease. The choice of disease to be treated depends largely on the nature of the nucleic acid sequence to be expressed by the expression cassette or vector of the invention. The isolated miR-206 target site of the invention allows to effectively exclude expression of said nucleic acid sequence in organs, tissues or cells with high level expression of miR-206 while expression of the transgene is maintained at high level in organs, tissue or cells with high level expression of miR-1. Since miR-1 is highly expressed in the heart, the method of the invention appears to be particularly suited in the treatment of heart diseases as coronary heart disease, cardiomyopathy, cardiovascular disease, ischaemic heart disease, heart failure, hypertensive heart disease, inflammatory heart disease, valvular heart disease.

The isolated miR-206 target site of the present invention allows silencing transgene expression in miR-206 expressing environment whereas in miR-1 expressing environment transgene expression is possible at high levels. Organ, tissue or cell-type specific expression of a transgene can further be modulated by combining the isolated miR-206 target site of the invention with other miR targeting sites. For example, Geisler et al. have already shown the potential of using miR-122 target sites to silence transgene expression in the liver. Thus, the present invention is also directed to an expression cassette of the invention and/or a vector of the invention further comprising at least one other miR target site, preferably at least one miR-122 target site.

In another aspect, the present invention is also directed to a method of modulating expression of a transgene, the method comprising the step of using an expression vector bearing a transgene to be expressed and at least one miR target site with at least one nucleotide substitution compared to the wild type miR target site. Preferably, the at least one miR target site is an isolated miR-206 target site of the invention. In a particular suitable embodiment, the miR target site is located in the 3′-UTR of the transgene to be expressed from the vector.

The present invention is also directed to the use of a miR target site with at least one nucleotide substitution compared to the respective wild type miR target site in the modulation of transgene expression. Preferably, the miR target site is an isolated miR-206 target site of the invention. In a particular suitable embodiment, the miR target site is located in the 3′-UTR of the transgene to be expressed from the vector.

### FIGURES

FIG. 1 relative miR-1, miR-206 and miR-122 expression levels determined by quantitative RT-PCR and effects of miRTS on transgene expression

A) Comparison of miR Expression In Vivo and In Vitro
Expression levels of miR-1, miR-206 and miR-122 in mouse liver, mouse heart, permanent mouse cardiac cell line HL-1, in C2C12 mouse myoblast and in neonatal mouse cardiomyocytes (NMCM) are shown as relatives to miR expression levels measured in mouse skeletal muscle. Significance: ***P<0.001; **P<0.01; *P<0.05 for each column compared to miR expression in muscle. Error bars represent means±S.E.M.; n=3 samples.
On the lower right panel PCR products to indicated PCR cycles of miR-1, miR-206 and miR-122 expression of indicated organs and cell lines are shown as agarose gel separated bands.

B) Sequences of miR-206 and miR-1
Sequence alignment of each skeletal muscle-specific miR-206 and muscle specific miR-1. Their conserved “seed” region is boxed and nucleotide consistency is marked by vertical lines.

C) miR Dependent Regulation of Firefly Luciferase Expression in Mouse Cardiac Myocytes and Mouse Myoblasts
HL-1, NMCM and C2C12 cells were transfected with a Firefly luciferase plasmid containing different miRTS. The ratio for cells transfected with luciferase construct with siHexTS (control) was set as 100% for each cell line transfected, and the other values for the same cell type were given relative to this reference. Firefly luciferase expression levels were normalized against Renilla luciferase expression levels in each sample. Significance: ***P<0.001; **P<0.01; *P<0.05 for each column versus control. Error bars represent means±S.E.M.; n=3 for each group.

FIG. 2 Effects of miR-206TS mutation on transgene expression in 293 cells expressing either miR-206 or miR-1

A) Sequences of Mutated miR-206 Target Sites
Sequence of single copy of siHexTS, miR-1TS, miR-206TS and mutated miR-206TS inserted into the 3′UTR of Renilla luciferase of psiCHECK-2 plasmid are highlighted in italics; restrictions sites are underlined. Capital letter indicates nucleotide substitution to parental miR-206TS. Region complementary to miR-1 and miR-206 seed region is boxed.

B) miR-206 and miR-1 Dependent Regulation of Luciferase Constructs Containing Mutated miR-206TS
293 cells were cotransfected with luciferase expression plasmid containing target sites shown under A) and plasmids expressing miR-206 and miR-1, respectively. Renilla luciferase expression levels were normalized against Firefly luciferase expression coexpressed from the same plasmid. The ratio for cells transfected with plasmid containing a target site and an unrelated miR (miR-122) was set as 100% and ratio for cells transfected with same TS and miR-206/miR-1 was given relative to this reference. Transfection of cells with plasmid containing a control sequence (siHexTS) and miR-122 vs. miR-206/miR-1 served as negative control. Significance was determined between plasmid containing siHexTS and miRTS and between plasmid with miR-206TS and mutated miR-206TS (m206TS), respectively. Significance differences are indicated. Significance: ***P<0.001; **P<0.01; *P<0.05. Error bars represent means±S.E.M.; n=3 individual experiments for each group, three replicates for one experiment.
   C) miR-206 and miR-1 Dependent Regulation of Luciferase Constructs Containing miR-206TS Mutation Located in the Sequence Complementary to miR Seed Sequence
293 cells were cotransfected with luciferase plasmid containing miR-206TS mutation located in the sequence complementary to miR seed sequence (shown under A) and miR-206 and miR-1, respectively. Measurement of luciferase expression levels, calculation of Renilla luciferase inhibition and determination of significant differences between investigated samples was performed as described under B.

D) miR-1 and miR-206 Dependent Regulation of Luciferase Constructs Containing Three Tandem Repeats of Chosen Mutated miR-206TS
293 cells were cotransfected with luciferase plasmid containing one copy of miR-1 and miR-206TS, one and three copy of chosen mutated TS m206TS-3G (m206(₁ₓ)TS and m206(₃ₓ)TS) and either miR-206 or miR-1. Measurement of luciferase expression levels and calculation of Renilla luciferase inhibition was performed as described under B. Significance differences are indicated. Significance: ***P<0.001; *P<0.05. Error bars represent means±S.E.M.; n=3 individual experiments for each group, three replicates for one experiment.

E) Degradation of Luciferase mRNA by miR-206 and miR-1
293 cells were transfected with luciferase plasmids containing one copy of the indicated control TS, miR-1TS, parental miR-206TS and one and three copies of the mutated TS m206TS-3G (m206(₁ₓ)TS and m206(₃ₓ)TS). Total RNA was isolated from cells 72 h after transfection. Expression levels of Renilla luciferase (hRluc) and Firefly luciferase (hluc+) mRNA were determined by Northern blotting using ³²P-labeled single stranded antisense probes directed against Renilla and Firefly luciferase, respectively. Compared to the control (luciferase plasmid coexpressed with miR-122), Renilla luciferase mRNA expression levels were reduced for cotransfection of miR-1TS and miR-206TS with miR-1 and miR-206 and for mutated miR-206TS with miR-206, but not with miR-1. Shown is a representative blot.

FIG. 3 Effects of miR-206TS mutation on transgene expression in skeletal muscle, heart and liver of mice

A) AAV Vector Design
Schematic of AAV vectors used for in vivo study. All AAV vectors contain the (CMV)-enhanced 0.26 kb MLC promoter (CMV-MLC0.26), the cDNA of human S100A1 with N-terminal FLAG-tag, the miRTS and the SV40pA signal; ITR, internal terminal repeats.

B) miR-122, miR-1 and miR-206 Dependent hS100A1 Expression in 293 Cells
293 cells were transfected with AAV constructs (FIG. 3A) and a plasmid expressing miR-122, miR-1 and miR-206, respectively. Control was a plasmid containing no miR(CMV wo miR). The putative regulated vectors contain inverted TS (invTS), three copies of miR-122TS plus three copies of mutated TS m206TS-3G (m206TS) and three copies of miR-122TS plus parental miR-206TS (TS). Transfected cells were harvested after 72 h and lysates probed with an S100A1 antibody. Upper lane contains samples of cells that were transfected in a ratio of 1:1 of plasmid versus miR; lower lane shows results of 1:3 transfection ratio. GAPDH served as internal load control.

C) AAV9 Mediated Transgene Expression in Different Tissues

Mice were injected with 1×10¹² vg of AAV9-hS100A1_{invTS} via vena jugularis injection. Animals were sacrificed after 16 days. RNA was isolated and expression of vector mediated human S100A1 in heart, liver, quadriceps femoris, tibia anterior, pancreas, lung, brain, spleen and kidney determined by quantitative RT-PCR. Analysis was carried out using the 2^{-ddCt} method and expression levels in organs were set relative to heart. Mouse HPRT expression was used for normalization. Significance: *P<0.05 for each column versus heart. Error bars represent means±S.E.M.; n=4 animals for each tissue.

D) Regulation of Transcription of AAV Mediated Transgene Expression in Heart, Skeletal Muscle and Liver by miRTS

Each five mice per group were injected with 1×10¹² vg of AAV9-hS100A1_{invTS}, AAV9-hS100A1_{m206TS} and AAV9-hS100A1_{TS} via vena jugularis. Organs were dissected after 16 days. RNA was isolated and expression of vector mediated hS100A1 in heart, liver and skeletal muscle (quadriceps femoris) was determined by quantitative RT-PCR. In each organ m RNA expression of AAV9-hS100A1_{m206TS} and AAV9-hS100A1_{TS} were set relative to expression mediated by AAV9-hS100A1_{invTS}, respectively. For each sample, mRNA abundance was normalized to amount of AAV DNA in the tissue. Significance: **P<0.01 for each column versus AAV9-hS100A1_{invTS} and **P<0.01; *P<0.05 for AAV9-hS100A1_{m206Ts} versus AAV9-hS100A1_{TS}. Error bars represent means±S.E.M.; n=5 animals for each tissue.
Expression of human S100A1 RNA was additionally analyzed by Northern Blotting using ³²P-labeled single stranded antisense probe directed against hS100A1, respectively. A representative blot with two mice per group is shown.

E) Regulation of Translation of AAV Mediated Transgene Expression in Heart, Skeletal Muscle and Liver by miRTS

Protein lysates of heart, liver and skeletal muscle (quadriceps femoris) were analyzed by ELISA using an antibody against S100A1 and Western Blot analysis using an antibody against FLAG-tag. For analysis of ELISA data, expression of hS100A1 protein of AAV9-hS100A1_{m206TS} and AAV9-hS100A1_{TS} were set relative to expression mediated by AAV9-hS100A1_{invTS} for each tissue. Protein expression was normalized to the amount of AAV vector DNA for each sample. Significance: **P<0.01; *P<0.05 for each column versus AAV9-hS100A1_{invTS} and **P<0.01; *P<0.05 for AAV9-hS100A1_{m206TS} versus AAV9-hS100A1_{TS}. Significance differences are indicated. Error bars represent means±S.E.M.; n=4 animals for each tissue.

### EXAMPLES

AAV vectors are currently the most specific and promising vector type used for gene transfer into the heart. However, these vectors also tranduce other organs, in particular the liver and the skeletal muscle. Recently we have shown that insertion of miR-122 specific target sites (TS) in the 3′UTR of a transgene is a powerful method to prevent transgene expression in the liver, see Geisler et al. Nevertheless selective suppression of cardiotropic AAV vector mediated transgene expression in skeletal muscle has not been shown yet. In this study we analyzed skeletal muscle specific miR-206 and miR-122 dependent transcriptional control of transgene expression in order to improve heart specificity of AAV-mediated gene transfer. Among miRs expressed in the heart and skeletal muscle, only miR-206 is highly expressed in skeletal muscle and rarely expressed in cardiac tissue. Application of AAV9 vectors bearing three complete complementary repeats of each, miR-122TS and miR-206TS, in the 3′UTR of a S100A1 cDNA resulted in the expected efficient silencing of transgene expression in the liver of mice. Surprisingly strong downregulation of recombinant S100A1 expression was also observed in the heart. In vitro analysis revealed that silencing in the heart was mediated by miR-1, which shows high homology (86%) to miR-206 and which is highly expressed in the heart and skeletal muscle. Hence we attempted to prevent miR-1 binding to miR-206TS without affecting miR-206 interaction to its TS by site-directed mutation of miR-206TS. Among 8 initially tested TS mutations, each containing one nucleotide substitution to the parental miR-206 TS, only one mutation (m206TS-3G) prevented suppression of reporter gene expression mediated by miR-1, accompanied by complete susceptibility to miR-206 regulation. Analysis of additional mutations revealed that only nucleotide substitutions (m206TS-2, -3A, -3T, -3G, -4, -5, -6, -7) located in the sequence complementary to the miR-206 seed sequence prevented miR-1 caused inhibition of transgene expression. Interestingly, several mutations (m206TS-4, -6, -7, -8, -12, -13, -14, -15, -16) of miR-206TS increased miR-206 mediated silencing of transgene expression compared to parental miR-206TS. Systemic application of AAV9 vectors containing S100A1 cDNA with three repetitive copies of the initially selected m206TS-3G resulted in a significant reduction of recombinant S100A1 mRNA and protein expression in skeletal muscle of mice. Recombinant S100A1 expression in the heart, however, was completely unaffected. Importantly, miR-206 and miR-1 expression levels were not affected in skeletal muscle and heart revealing that mutation of miR-206TS did not affect miR expression. In conclusion, we have developed a new miR regulated AAV vector with increased cardiac specificity and have demonstrated for the first time that mutation of miRTS is a suitable approach to parry “undesirable” miRs. Furthermore, we have shown that site direct mutation of a miRTS can result in enhancement of TS mediated inhibition of transgene expression These findings may open new ways to increase tissue specificity of vectors for use in gene therapy.

### Results

miR-206 is Exclusively Expressed in Skeletal Muscle and in C2C12 Myoblasts

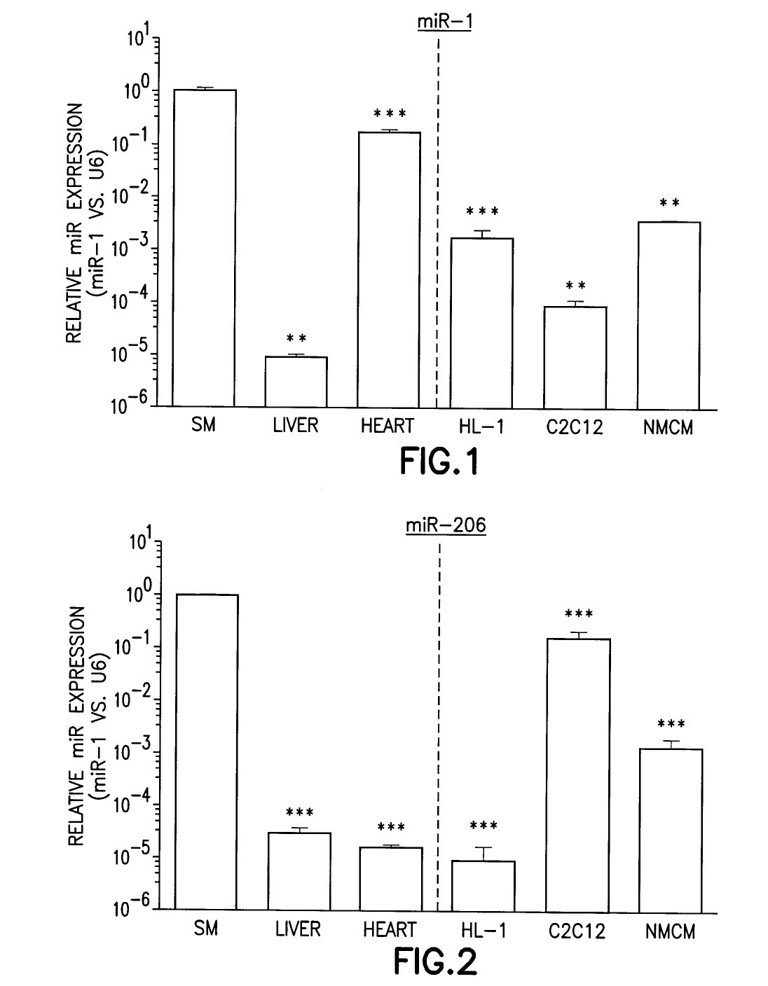

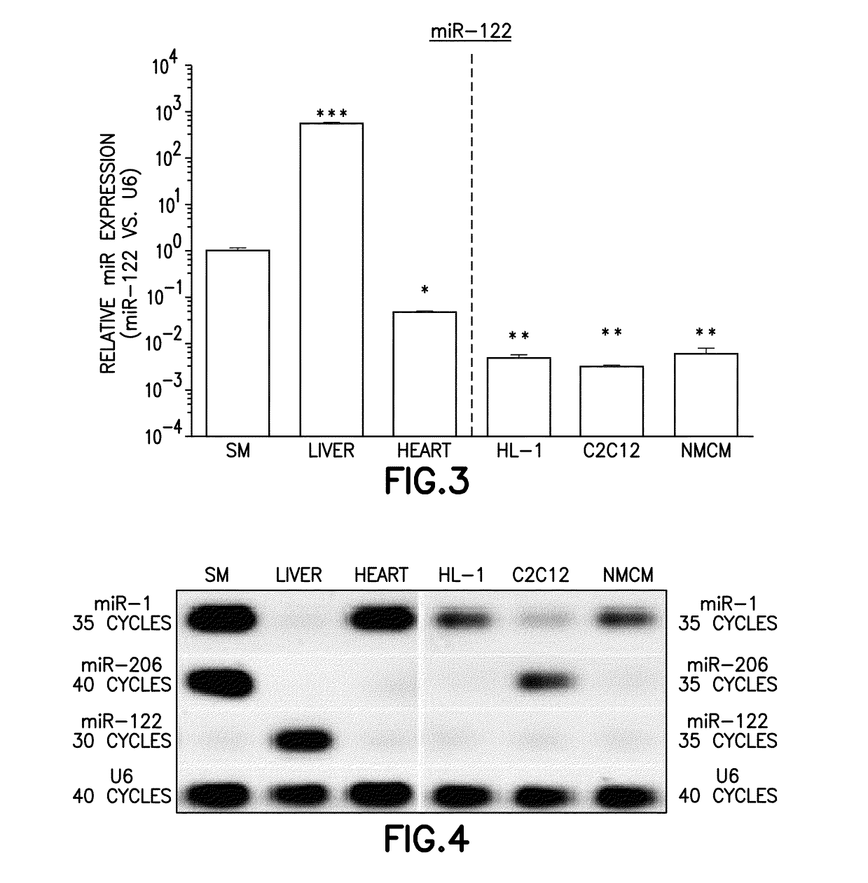

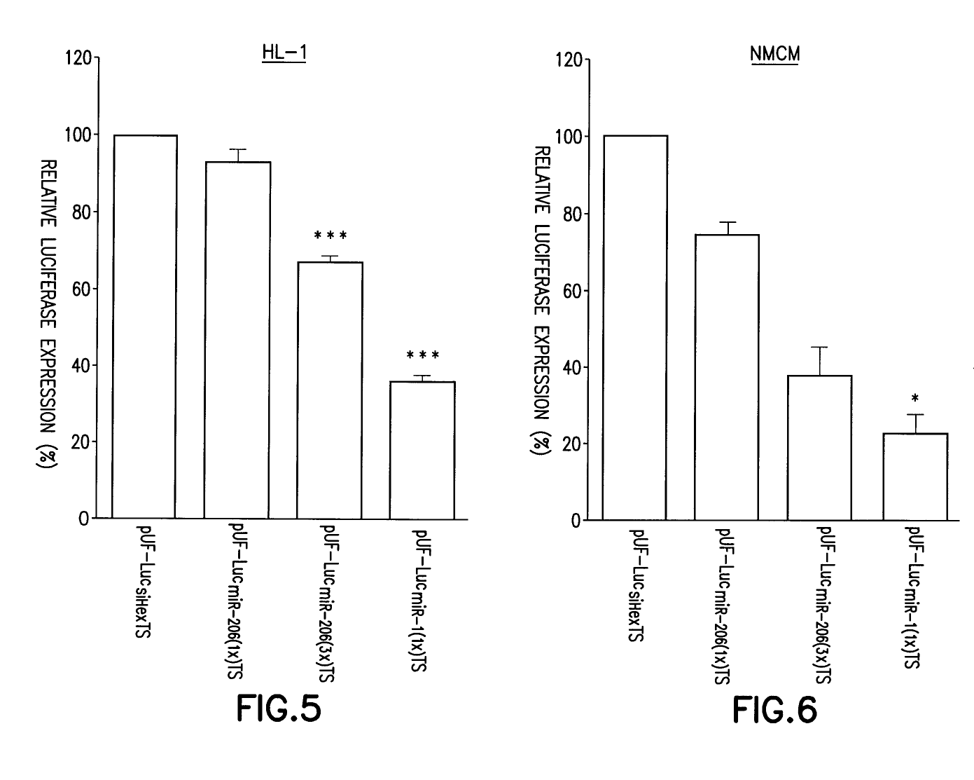

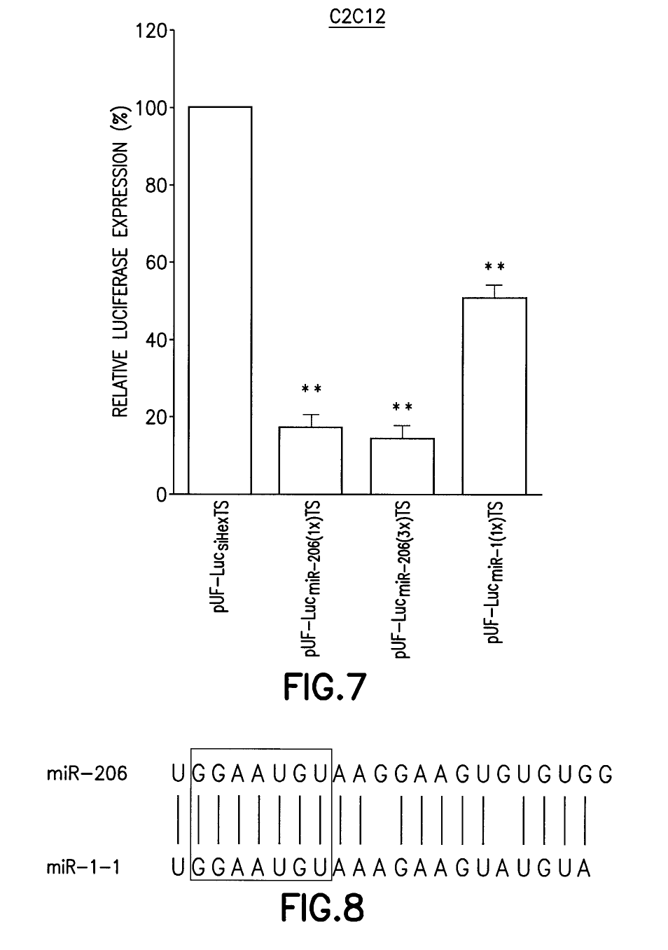

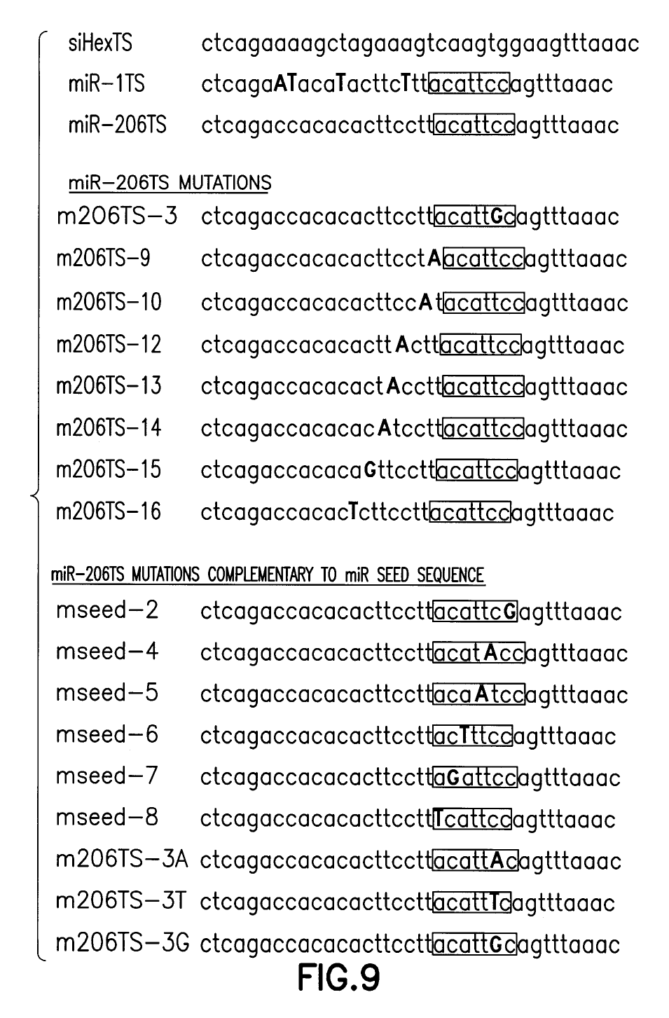

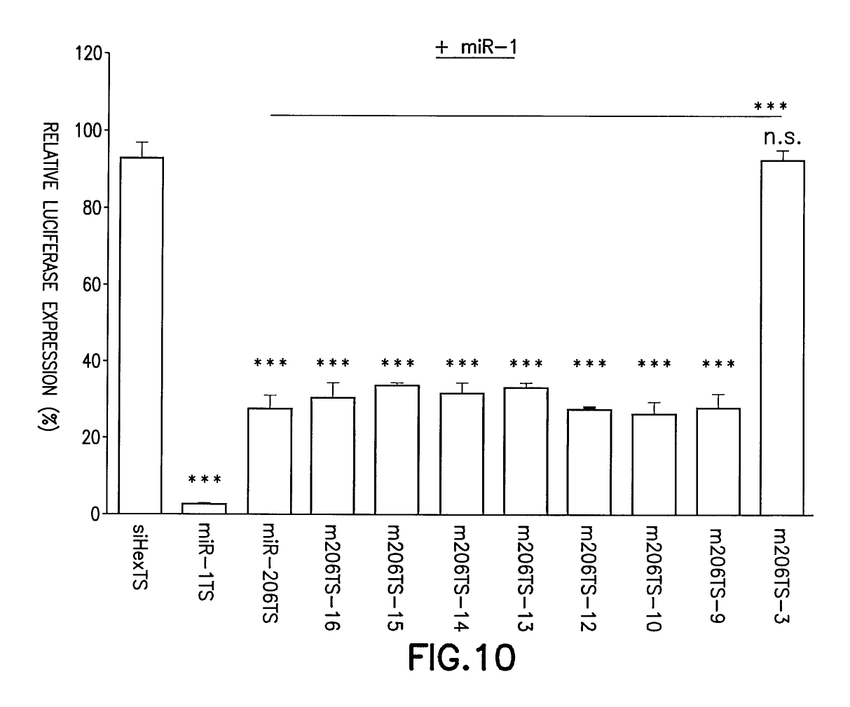

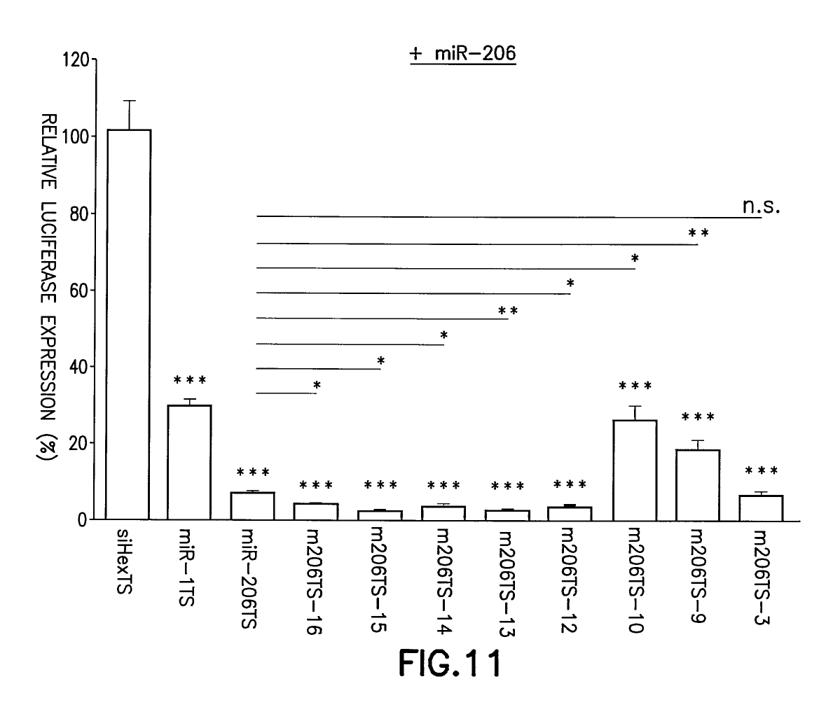

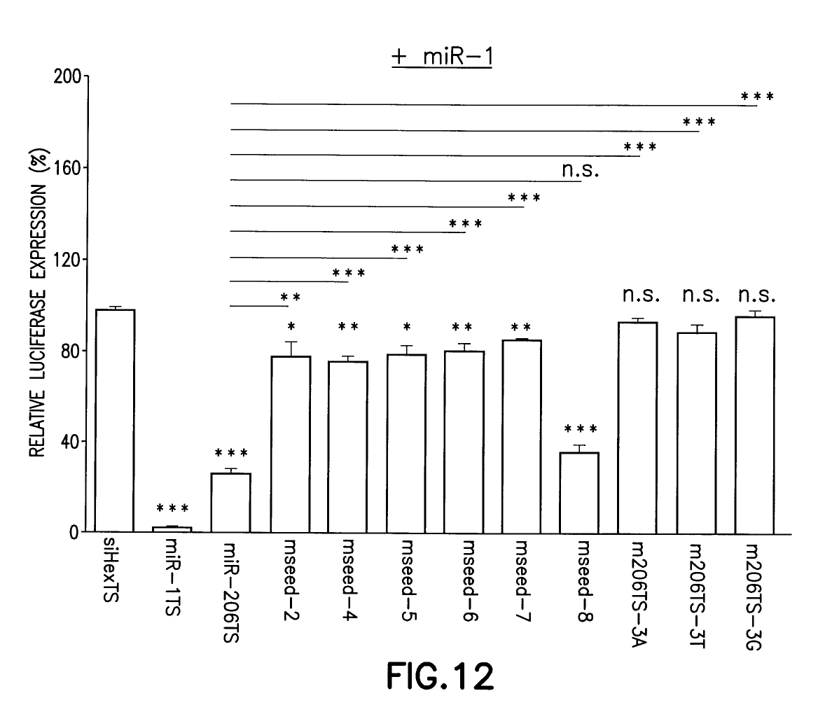

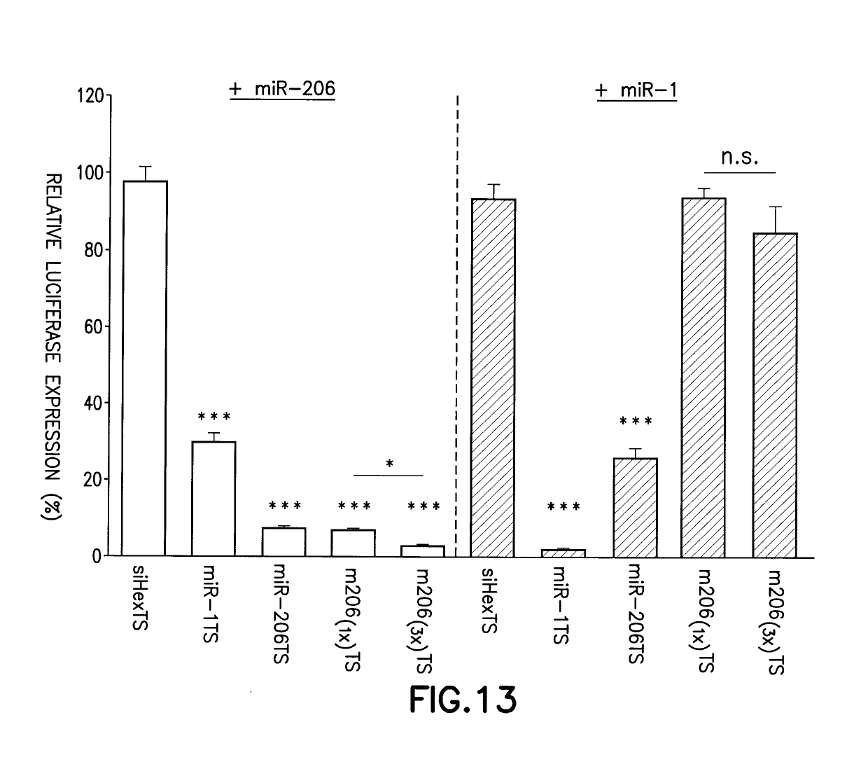

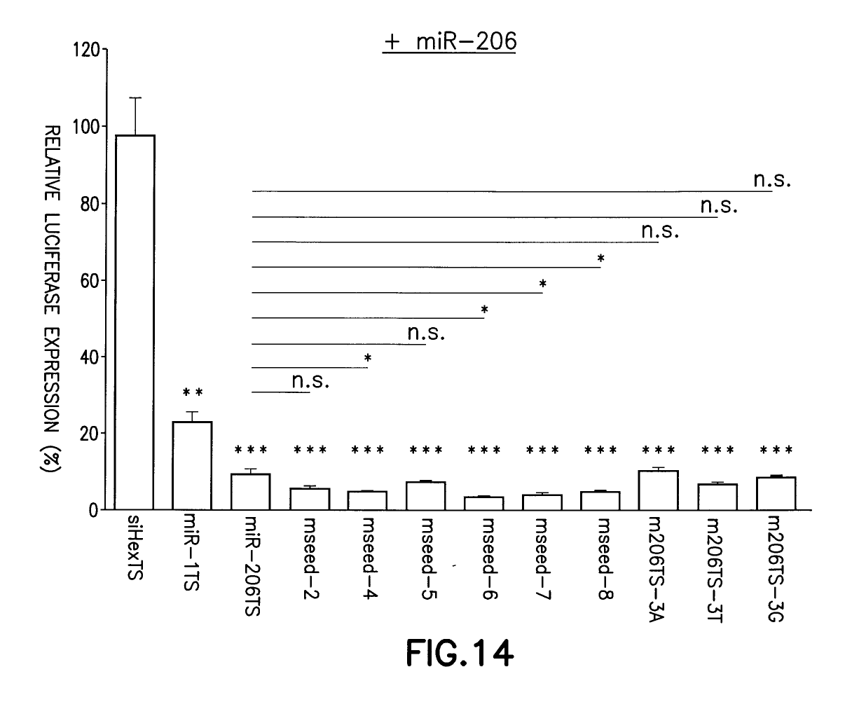

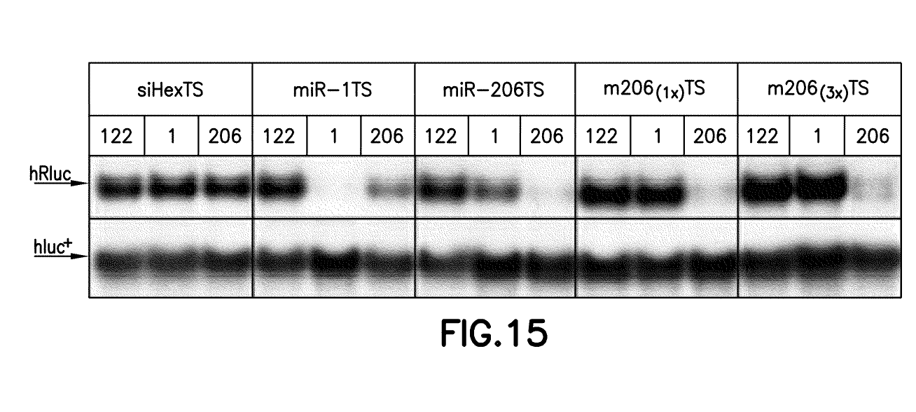

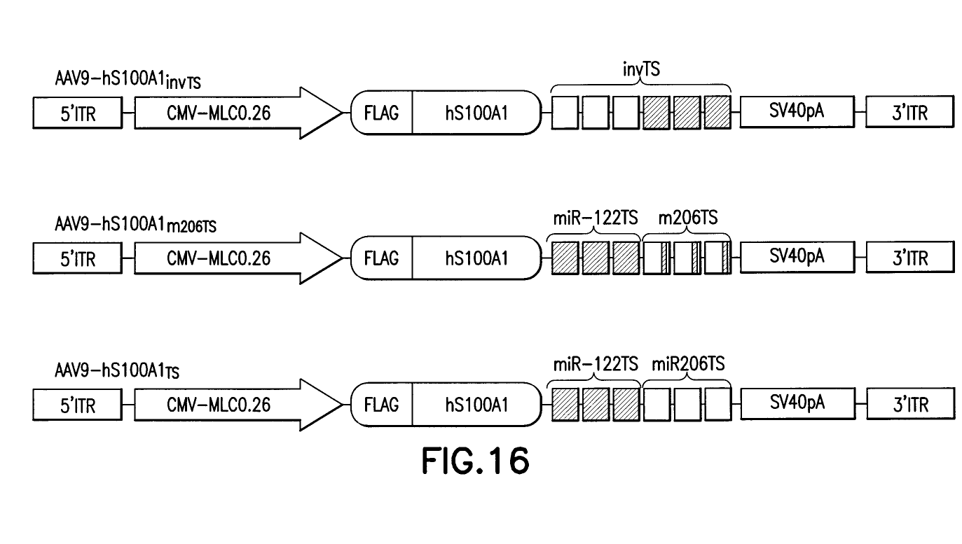

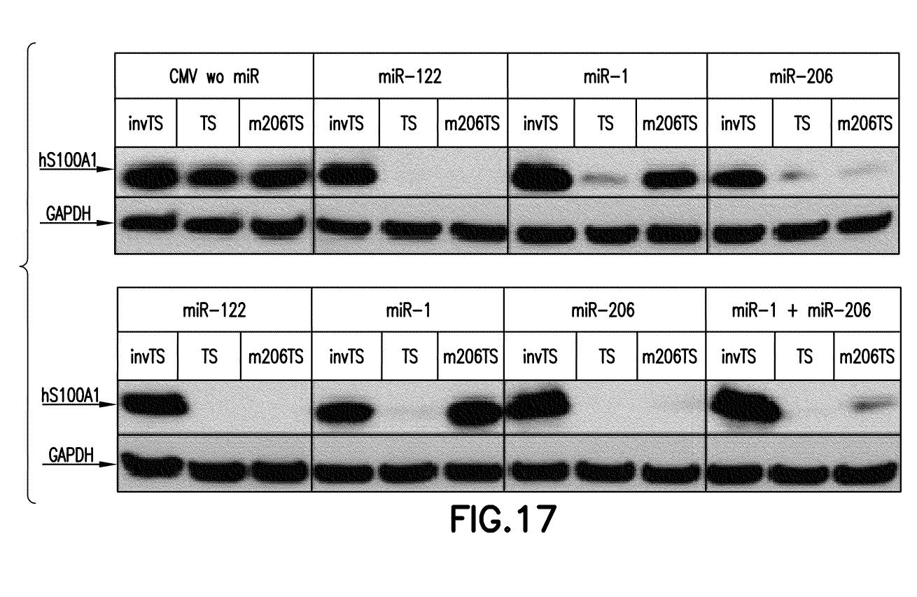

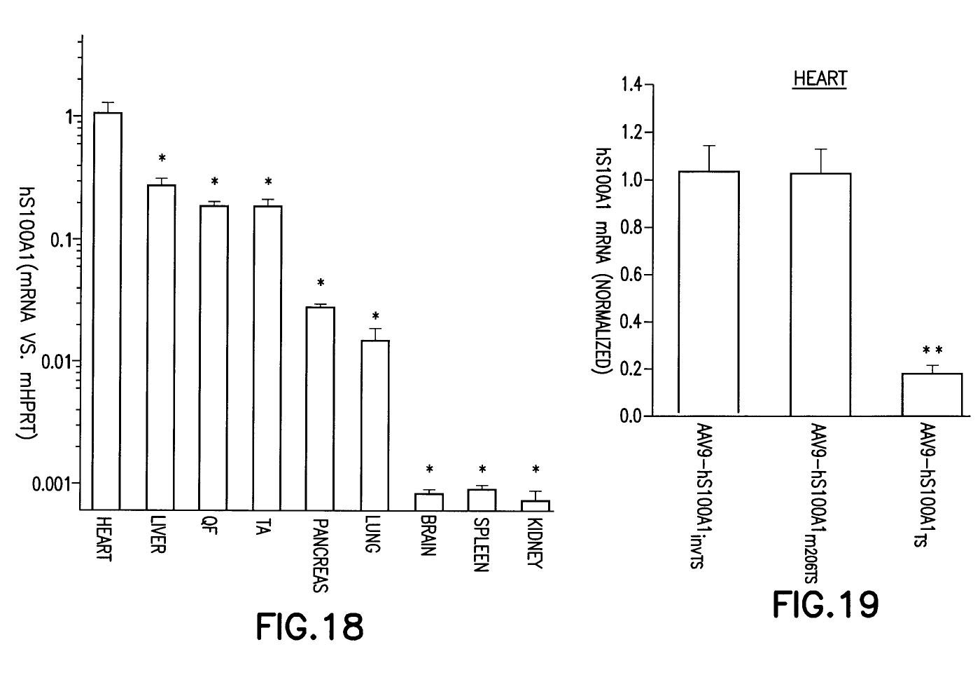

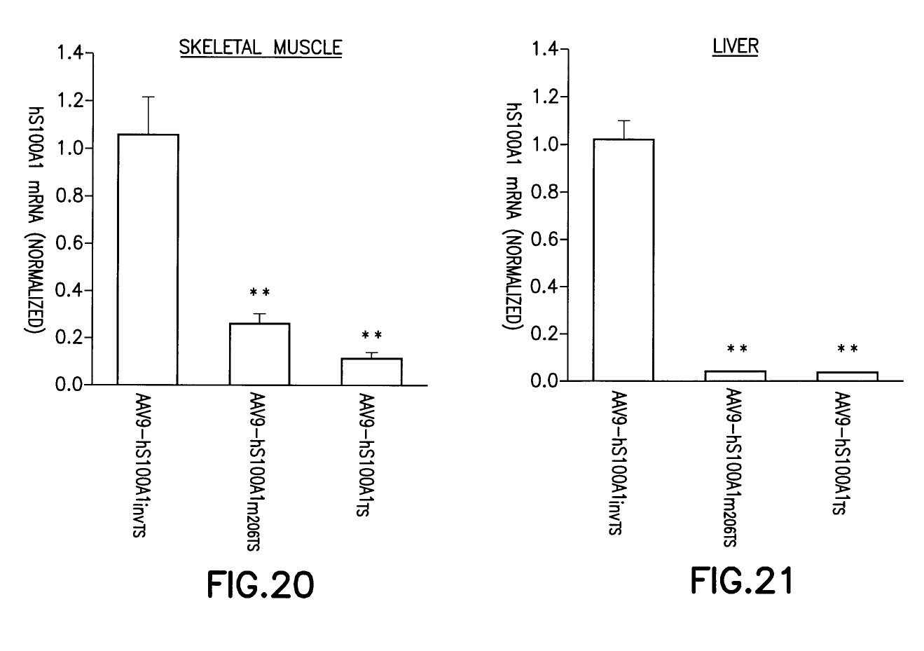

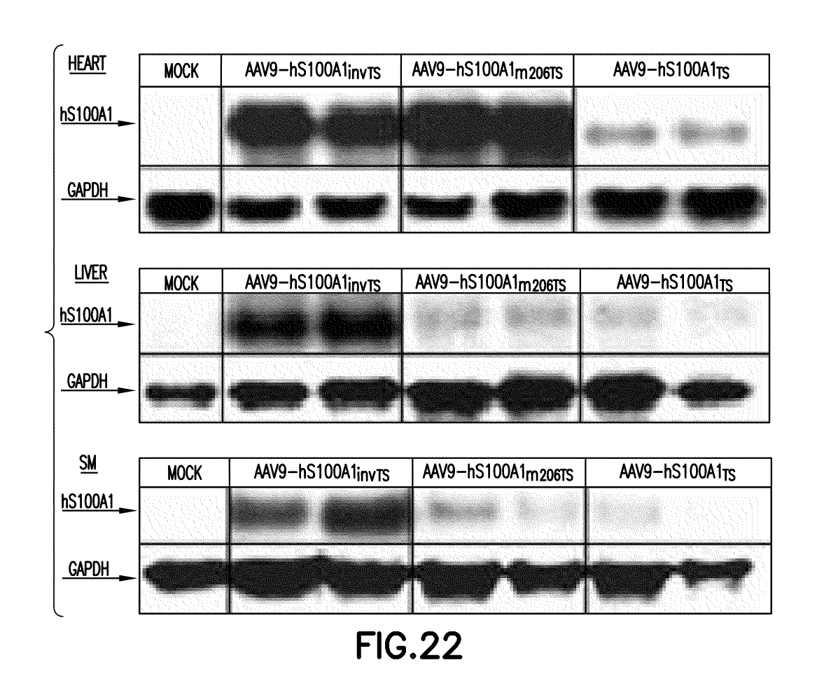

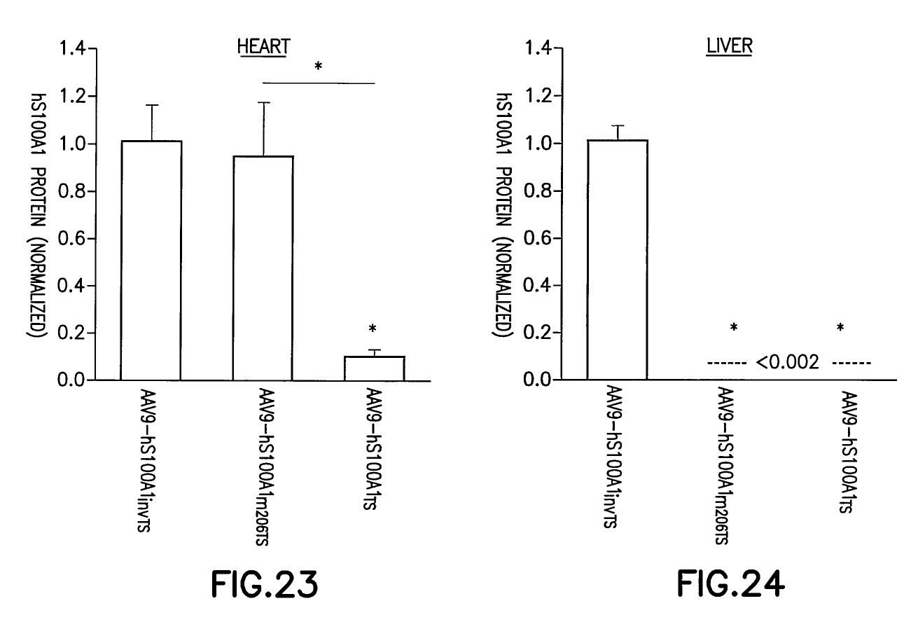

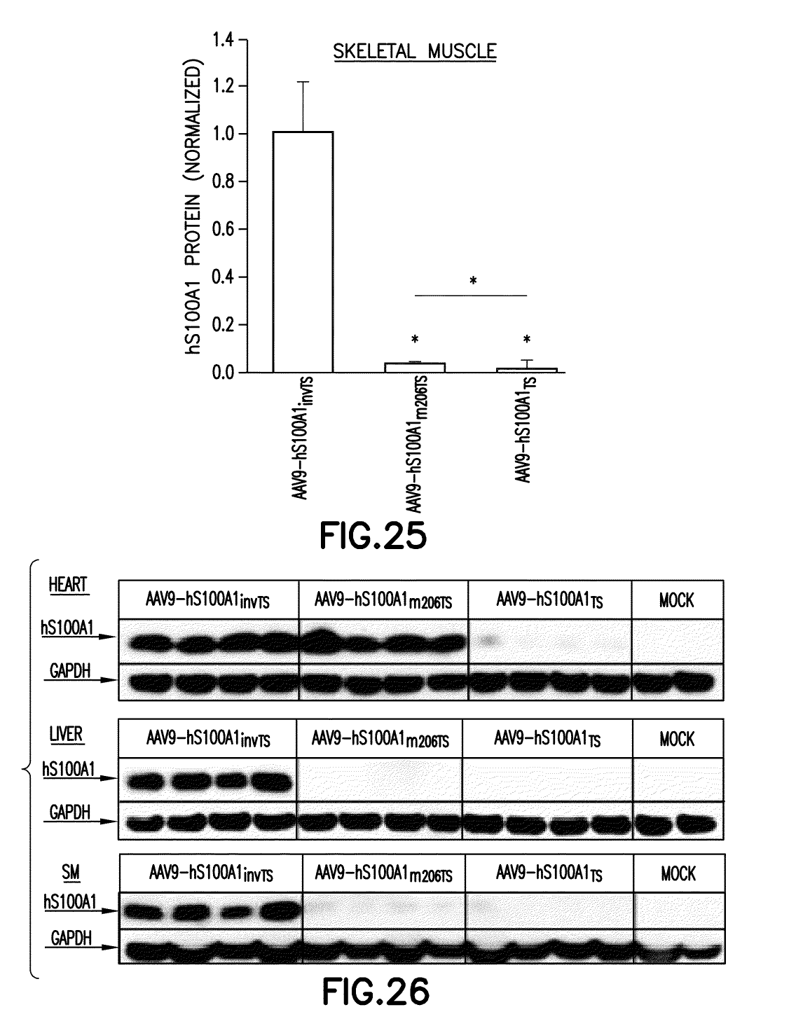

To quantify the specific expression of known miRs in AAV targeted mouse tissues, we assayed the expression of miR-1, miR-206 and miR-122 in skeletal muscle, liver and heart and in cardiac and skeletal muscle derived cell lines and neonatal mouse cardiomyocytes (NMCM) (FIG. 1A). miR-1 was highly expressed in mouse skeletal muscle and mouse heart, and at lower levels in HL-1 cells, a cell line derived from murine atrium, and NMCM. Low expression was observed for C2C12 and almost no expression was seen in mouse liver. miR-206 was exclusively found in skeletal muscle and in mouse C2C12 myoblasts and miR-122 expression was almost restricted to liver. Due to the high homology of miR-1 and miR-206 (FIG. 1B), we wanted to test the effects of miR-206 or miR-1 target sites insertion on transgene expression in miR-206 or miR-1 expressing cells. In HL-1 cells and in NMCM, where miR-1 was expressed at moderate levels, insertion of one copy of miR-1TS into the 3′UTR of a luciferase expression cassette, led to a reduction of transgene expression (up to 64% in HL-1 and 77% in NMCM, FIG. 1C). Both cell lines expressed miR-206 at very low levels. Insertion of at least three repeats of miR-206TS caused a reduction of luciferase activity up to 34% in HL-1 and moreover up to 66% in NMCM, indicating a miR-1 caused inhibition of reporter gene expression. Transfection of luciferase reporter containing one and three copies of miR-206TS in C2C12 cells reduced reporter activity up 85% and 89% caused by high expression levels of miR-206.

Mutation of miR-206TS Abrogates miR-1 Suppression Activity

In order to abolish miR-1 mediated inhibition of transgene expression without affecting suppression by miR-206 and to further increase cardiac AAV mediated transgene transfer, we engineered several miR-206TS mutation each containing one nucleotide substitution complementary to parental miR-206TS (FIG. 2A miR-206TS mutations). Coexpression of miR-206 and luciferase reporter with parental miR-206TS led as expected to inhibition of luciferase activity up to 93% compared to control (FIG. 2B lower panel). Stronger suppression with up to 97% was observed for coexpression of miR-1 and miR-1TS (FIG. 2B upper panel). Cross-reactivity of miR-1 and miR-206TS and miR-206 with miR-1TS led to a similar reduction of reporter activity with up to −30%. Modification of nucleotide position 9 to 16 of miR-206TS did not alter miR-1 mediated inhibition of reporter activity compared to downregulation of parental miR-206TS by miR-1 (FIG. 2B upper panel). Interestingly miR-206TS mutation on position 12 to 16 led to a significant stronger suppression of transgene expression by miR-206 compared to parental miR-206 (FIG. 2B lower panel). A significant decline of miR-206 suppression compared to parental miR-206 was observed for change of nucleotide position 9 and 10. Surprisingly only modification of nucleotide position 3, a position located in the sequence complementary to miR-1 and miR-206 seed region, led to prevention of miR-1 mediated silencing of transgene expression without affecting suppression by miR-206 (FIG. 2B).

To investigate whether a modification of each single nucleotide in the sequence complementary to miR-1 and miR-206 seed region was suitable to prevent a miR-1 caused inhibition of transgene expression, we constructed several miR-206TS variants with modifications within this short part of TS (FIG. 2A). Except for the miR-206TS containing a nucleotide substitution on position 8, all constructs were able to abolish miR-1 mediated suppression of reporter activity (FIG. 2C upper panel). Nucleotide modification on positions 2 to 7 were little less effective than on position 3. Parental miR-206TS contains a cytosine on nucleotide position 3. Substitution of cytosine with adenosine, thymine or guanine had the same potential to prevent miR-1 caused inhibition of transgene expression and had no influence on miR-206 mediated suppression of luciferase activity compared to parental TS (FIG. 2C). Although mutations of nucleotide position 4, 6, 7 and 8 showed a significant increase in miR-206 mediated inhibition of reporter expression compared to parental miR-206TS (FIG. 2C lower panel), the mutated miR-206TS with nucleotide guanine on position 3 (m206TS-3G) showing strongest abrogation of miR-1 suppression activity, was chosen for further in vitro and in vivo studies. Hence we tested whether an increasing number of mutated miR-206TS copies had an effect on miR-1 and miR-206 mediated transgene expression. Cotransfection of 293 cells with luciferase reporter containing a triplet of chosen mutated miR-206TS (m206(₃ₓ)TS) and miR-206 resulted in stronger suppression of reporter activity compared to reporter containing a single mutated miR-206TS (m206(₁ₓ)TS) whereas abolishment of miR-1 activity still remained unchanged (FIG. 2D).

In order to investigate whether miR mediated inhibition of reporter gene expression resulted from mRNA degradation or inhibition of protein translation, we analyzed Renilla luciferase mRNA expression levels by Northern hybridization. Compared to control transfection with miR-122, mRNA levels of parental miR-206TS, miR-1TS and mutated miR-206TS were distinctly decreased for cotransfection with miR-206 and mRNA levels of miR-206TS and miR-1TS for cotransfection with miR-1, indicating that regulation of reporter gene expression preferentially occur through mRNA degradation (FIG. 2E). Corresponding to luciferase measurements, mRNA degradation of parental miR-206TS caused by miR-1 and miR-1TS by miR-206 occurred at a less degree and no differences in mRNA levels of mutated miR-206TS with miR-1 coexpression compared to control could be observed.

Mutated miR-206TS Regulated AAV9 Vectors Detarget the Skeletal Muscle without Affecting Cardiac Transgene Expression in Mice

Initially, we tested AAV9 shuttle plasmid expressing human S100A1 under the control of the (CMV)-enhanced 0.26 kb MLC promoter (CMV-MLC0.26) and containing either the parental miR-206_{(3X)}TS (AAV9-hS100A1_{TS}) or the mutated miR-206_{(3X)}TS-3G (AAV9-hS100A1_{m206TS}) (FIG. 3A). Additionally both vectors contained a triplet of miR-122TS to detarget AAV mediated expression from liver. By insertion of inverted miR-206₍₃ₓ₎TS and miR-122_{(3X)}TS, we created a no miR regulated control vector (AAV9-hS100A1_{invTS}). Cotransfection of 293 cells with AAV shuttle plasmids and either miR-122 (control), miR-1, miR-206 or miR-1+ miR-206 resulted in a miR dependent specific inhibition of hS100A1 protein expression (FIG. 3B).

16 days after administration of self complementary AAV9 vectors, we analyzed mRNA expression levels of human S100A1 in different tissues involving heart, liver, quadriceps femoris, tibia anterior, pancreas, lung, brain, spleen and kidney (FIG. 3C). Compared to heart, relative high expression of hS100A1 was measured for liver with ˜30% and in skeletal muscle with ˜18%. Expression of AAV mediated transgene expression in pancreas and lung reached levels below 3% and in brain, spleen and kidney below 0.1%.

Expression levels of human S100A1 were assayed in detail for heart, liver and skeletal muscle using quantitative reverse transcription PCR (qRT-PCR). For the heart, we observed an ˜87% reduction of transcription levels for AAV9 vectors containing the parental miR-206_{(3X)}TS compared to vectors with inverted TS (control) (FIG. 3D). In contrast, no reduction was found for AAV9 vectors expressing hS100A1 containing the mutated miR-206₍₃ₓ₎TS.

### Materials and Methods

### Plasmid Construction

AAV shuttle plasmids containing a single miR-1TS (pUF-Luc_{miR-1(1x)TS}) and miR-206TS (pUF-Luc_{miR-206(1x)TS}), a triplet of the miR-206TS (pUF-Luc_{miR-206(3x)TS}) and a non related target sequence from adenovirus hexon protein (pUF-Luc_{siHexTS}) were derived from initial plasmid pUF-CMVₑₙₕ/MLC0.26-Luc containing a cardiac (CMV)-enhanced 0.26 kb rat myosin light chain promoter (MLC0.26) driving a firefly luciferase reporter gene (Müller et al., 2003; Geisler et al., 2010). Target sequences were placed between the luciferase cDNA and the SV40 polyA signal. Single copy of miR-1TS fragment was generated by annealing primers 5′-ctagtatacatacttctttacattccat-3′ (SEQ ID No. 23) and 5′-ctagatggaatgtaaagaagtatgtata-3′ (SEQ ID No. 24) and insertion into pUF-CMVₑₙₕ/MLC0.26-Luc via a single XbaI site at the 3′UTR. For generation of pUF-Luc_{miR-206(1x)TS} the primers 5′-ctaggccacacacttccttacattccat-3′ (SEQ ID No. 25) and 5′-ctagatggaatgtaaggaagtgtgtggc-3′ (SEQ ID No. 26) were annealed and ligated into XbaI linearized initial plasmid. The oligonucleotides 5′-cgaaagctagaaagtcaagtggaat-3′ (SEQ ID No. 27) and 5′-ctagattccacttgactttctagctttcg-3′ (SEQ ID No. 28) were annealed and ligated into NruI and XbaI digested pUF-Luc_{miR-122(3x)TS/miR-148a(1x)TS} (Geisler et al., 2010) resulting in pUF-Luc_{siHexTS}. The plasmid pUF-Luc_{miR122(3x)TS/miR148a(1x)TS} was digested with StuI and XbaI removing miR-148a fragment and inserting miR-206TS fragment of annealed primers 5′-cctccacacacttccttacattccat-3′(SEQ ID No. 29) and 5′-ctagatggaatgtaaggaagtgtgtggagg-3′ (SEQ ID No. 30) resulting in pUF-Luc_{miR-122(3x)TS/miR-206(1x)TS}. Second and third copy of miR-206TS were stepwise generated by annealing of 5′-ctaggccacacacttccttacattccat-3′ (SEQ ID No. 31) and 5′-ctagatggaatgtaaggaagtgtgtggc-3′ (SEQ ID No. 32) and ligation into XbaI digested pUF-Luc_{miR-122(3x)TS/miR-206(1x)TS} resulting in pUF-Luc_{miR-122(3x)TS/miR-206(3x)TS}, respectively. By digestion with NruI and StuI, miR-122₍₃ₓ₎TS fragment was removed and plasmid religated resulting in pUF-Luc_{miR-206(3x)TS}.

psiCHECK-2 plasmids (Promega, Mannheim, Germany) containing miRTS (FIG. 2A) were generated by insertion of appropriate DNA fragments obtained by oligonucleotide annealing into the 3′UTR of Renilla luciferase cDNA of psiCHECK-2 via XhoI and PmeI restriction site. To generate psiCHECK-2-m206₍₃ₓ₎ TS, a synthesized DNA fragment containing three repeats of mutated miR-206TS (aggcctccacacacttccttacattgcatctaggccacacacttccttacattgcatctaggccacacacttccttacattgcatctag a-3′; SEQ ID No. 33; GeneArt, Life Technologies, Darmstadt, Germany) was digested with StuI and XbaI, 5′ overhangs were filled in and fragment ligated into XhoI and PmeI restricted and 5′ filled-in psiCHECK-2 plasmid.

To express miR-206 and miR-122, the plasmids miR-Vec-miR-206 (miR-206) and miR-Vec-miR-122 (miR-122) were used (kind gift from Alexander Karlas, Max Planck Institute for Infection Biology, Berlin, Germany). These plasmids contain miRNA minigenes that were amplified by PCR from genomic human DNA and cloned downstream of the CMV promoter in miR-Vec (Voorhoeve and Sage, 2006). To generate miR-Vec-miR-1 (miR-1), a sequence from miRNASelect™pEGFP-mmu-mir-1-2 Expression Vector (Cell Biolabs Inc., San Diego, USA) was cloned into miR-Vec-miR-206 via BamHI and EcoRI restriction sites resulting in miR-Vec-miR-1. Restriction of miR-Vec-miR-206 via BamHI and EcoRI, 5′ filled in and religation led to a plasmid expressing no miR(CMV wo miR).

Initial AAV vector shuttle plasmid pscAAV-CMV-MLC0.26-S100A1 with self complementary vector genome containing a β-globin/IgG chimeric intron and the cDNA for human S100A1 under control of the CMV-MLC0.26 promoter was a kind gift of 0. Müller (DKFZ, Heidelberg, Germany). To insert miRTS into the 3′UTR of pscAAV-CMV-MLC0.26-S100A1 a fragment containing miR-122₍₃ₓ₎TS and parental miR-206₍₃ₓ₎TS was amplified from pUF-Luc_{miR-122(3x)TS/miR-206(3x)TS} using primers 5′-ccagtgtacatcgcgaacaaac-3′ (SEQ ID No. 34) and 5′-ataatgtacaggccgccccgactcta-3′ (SEQ ID No. 35). PCR product was inserted into single BsrGI restriction site resulting in pscAAV-CMV-MLC0.26-S100A1_{miR-122(3x)TS/miR-206(3x)TS} and pscAAV-CMV-MLC0.26-S100A1_{invTS} dependent on orientation of insertion. To amplify the sequence of human S100A1 containing a FLAG-tag at the N-terminus, two successive PCRs for each plasmid were run. PCR product of pscAAV-CMV-MLC0.26-S100A1_{miR-122(3x)TS/miR-206(3x)TS}, amplified with primer pairs P1 NF-S100A1 sense 5′-aaagacgatgacgacaagggctctgagctggagacgg-3′ (SEQ ID No. 36) and P1 NF-S100A1-TS NruI antisense 5′-aagttcgcgagtcaactgttctcccag-3′ (SEQ ID No. 37); P2 NF-S100A1 s 5′-aatcggatccatggactacaaagacgatgacgacaag-3′ (SEQ ID No. 38) and P1 NF-S100A1-TS NruI antisense 5′-aagttcgcgagtcaactgttctcccag-3′ (SEQ ID No. 39) was inserted into BamHI and NruI restriction site of pscAAV-CMV-MLC0.26-S100A1_{miR-122(3x)TS/miR-206(3x)TS} generating pscAAV-CMV-MLC0.26-NFS100A1_{miR-122(3x)TS/miR-206(3x)TS} (AAV9-hS00A1_{invTS}). PCR conditions to generate pscAAV-CMV-MLC0.26-NFS100A1_{invTS} (AAV9-hS00A1_{invTS}) were similar except that primer P1 NF-S100A1-TS NruI antisense was changed by P1 NF-S100A1-aTS XbaI antisense 5′-aagttctagagtcaactgttctcccag-3′ (SEQ ID No. 40). PCR product was cloned into BamHI and XbaI restriction site of pscAAV-CMV-MLC0.26-S100A1_{invTS}. To exchange parental miR-206₍₃ₓ₎TS by the mutated miR-206₍₃ₓ₎TS-3G, the synthesized DNA fragment containing three repeats of mutated miR-206TS-3G (aggcctccacacacttccttacattgcatctaggccacacacttccttacattgcatctaggccacacacttccttacattgcatctag a-3′ (SEQ ID No. 41); GeneArt, Life Technologies, Germany) was inserted into StuI and XbaI sites of AAV9-hS00A1_{TS} resulting in pscAAV-CMV-MLC0.26-NFS100A1_{miR-122(3x)TS/m206(3x)TS-3G} (AAV9-hS00A1_{m206TS}). Plasmids were controlled by sequence analysis using an ABI 310 Genetic Analyzer (Applied Biosystems, Life Technologies).

### Generation of AAV Vectors

scAAV9 vectors used in this stuy were generated, purified and titered as described previously (Geisler et al., 2010) with some variations in transfection procedure. Briefly, 293 cells were seeded in collagen (Calbiochem, Merck KgaA, Darmstadt, Germany) coated roller bottles (Corning, Thermo Fisher Scientific Inc., Rockford, Ill., USA) and transfected at next day at a confluence of about 70%. For transfection of one bottle, 593.40 μg Polyethylenimine (PEI, Polysciences, Inc., Warrington, Pa., USA) was diluted in 7 ml of 150 mM NaCl and 50 μg AAV shuttle plasmid, 90 μg of p5E18VD2/9 (kind gift of J. M. Wilson, University of Pennsylvania, PA, USA) and 90 μg of pHelper (Agilent Technologies, Inc., Santa Clara, Calif., USA) were diluted in 7 ml of 150 mM NaCl. PEI solution was than added dropwise to the plasmid solution and incubated at room temperature for 15 min. The transfection mixture was added dropwise into the bottle with 140 ml medium and cells were incubated at 37° C. After an incubation period of 72 h, cells were harvested. Cells were further washed and prepared for purification carried out by a filtration cascade followed by an iodixanol step gradient centrifugation as described previously (Geisler et al., 2010).

### Cell Culture

293 cells (human embryonal kidney) and C2C12 myoblasts were cultured in high glucose Dulbecco's Modified Eagle's Medium (DMEM, Gibco, Life Technologies) supplemented with 10% fetal calf serum (FCS) and 50 μg/ml both penicillin and streptomycin. The HL-1 cell line, a cardiac muscle cell line established from an AT-1 mouse atrial cardiomyocyte tumor lineage, was a kind gift from William C. Claycomb (LSU Health Center, New Orleans, USA). The cells were maintained in Claycomb medium (SAFC Biosiences, Kansas, USA) supplemented with 10% FCS, 1% each of penicillin and streptomycin and 2 mM L-glutamine (Invitrogen, Life Technologies). Before culturing HL-1 cells, tissue culture flasks were coated with 0.02% gelatine/fibronectin. Primary neonatal mouse cardiomyocytes (NMCM) were isolated from neonatal mice, plated in DMEM with 17% Medium 199 (Sigma, Sigma-Aldrich, Steinheim, Germany), 10% horse serum, 5% fetal bovine serum (FBS; PAA Laboratories GmbH, Pasching, Austria) and maintained in DMEM with 20% Medium 199 (Sigma, Sigma-Aldrich), 1% horse serum (HS, Biochrom AG, Berlin, Germany), 2 μM 5′ Fluoro-2′-deoxyuridine (Sigma, Sigma-Aldrich, Germany) and 50 μg/ml both penicillin and streptomycin.

### Plasmid Transfection

Plasmids were transiently transfected into HL-1 cells using Lipofectamine 2000 (Invitrogen, Life Technologies) and into NMCM and C2C12 using Lipofectamine LTX+ Plus Reagent (Invitrogen, Life Technologies) in accordance with the manufacturer's instructions. 24 h after seeding in 24-well plates, cells were transfected with 100 ng of plasmids expressing Firefly luciferase (pUF-Luc) with non related TS (siHexTS) or miRTS, 10 ng plasmid encoding Renilla luciferase for standardisation and 690 ng of an unrelated carrier plasmid containing a green fluorescent protein cDNA. 2 days before seeding of C2C12, nearly dense cells were switched into DMEM containing 2% heat inactivated HS. After transfection procedure, medium was changed by fresh DMEM containing 2% HS. 293 cells, seeded in 48-well cell culture plates, were transfected with 50 ng psiCHECK-2 plasmid and 350 ng of miR-Vec plasmid using PEI transfection reagent. 293 cells, seeded in 12-well culture plates, were transfected with 1 μg pscAAV-CMV-MLC0.26-NFS100A1_{invTS}, pscAAV-CMV-MLC0.26-NFS100A1_{miR-122(3x)TS/miR-206(3x)TS}, pscAAV-CMV-MLC0.26-NFS100A1_{miR-122(3x)TS/m206(3x)TS-3G} and 1 μg or 3 μg miR-Vec plasmid, respectively, using PEI transfection reagent. Analysis of reporter gene expression was carried out 72 h after transfection.

### Reporter Assay

Firefly and Renilla luciferase activity were measured using the Dual-Luciferase Reporter Assay (Promega GmbH, Mannheim, Germany) according to manufacturer's instructions. Luciferase activity was measured in a Lumat LB 9507 luminometer (Berthold Technologies, Bad Wildbad, Germany).

qRT-PCR Analysis

Total RNA from animal tissue was isolated using Trizol (Invitrogen, Life Technologies) and RNA from cultured cells with mirVana™ miRNA Isolation Kit (Ambion, Life Technologies) according to the recommendation of the supplier. Total RNA (0.5 μg) was digested with DNaseI (peqlab Biotechnologie GmbH, Erlangen, Germany) and reverse transcribed (High Capacity cDNA Reverse Transcription Kit, Applied Biosystems, Life Technologies) using Oligo-d(T)₁₆ primers (Applied Biosystems, Life Technologies). Expression of human S100A1 was determined by real time PCR using oligonucleotide primers/probe as follows: hS100A1s 5′-gggctctgagctggagacg-3′ (SEQ ID No. 42), hS100A1 as 5′-caccttgtccacagcatcca-3′ (SEQ ID No. 43), hS100A1 probe 6FAM-ccactcgggcaaagaggggg-TAMRA (SEQ ID No. 44). mRNA abundance was normalized to vector DNA detected in each sample. Mouse HPRT was used for normalization of mRNA expression (TaqMan Gene Expression Assay mHPRT1, Applied Biosystems, Life Technologies). Expression levels of murine Gys1 and Connexin43 were determined by real time PCR (TaqMan Gene Expression Assay mouse Gys1 and Connexin43 (Applied Biosystems, Life Technologies). To analyse expression of mature miRs, 10 ng of isolated RNA was reverse transcribed using miR-122, miR-1 and miR-206 specific primers and expression levels were determined by real time PCR (TaqMan MicroRNA Assays hsa-miR-122, hsa-miR-1, hsa-miR-206, Applied Biosystems, Life Technolgies). For normalization, U6snRNA expression was assayed (Applied Biosystems, Life Technolgies). Quantitative PCR reactions were carried out in triplicate using the TaqMan Gene Expression Mastermix (Applied Biosystems, Life Technologies), the Bio-rad C1000™ Thermal Cycler and CFX96™ Real-Time-System (Bio-rad Laboratories, München, Germany).

### Northern Blot Analysis

To detect human S100A1, 5 μg of isolated total RNA of heart and 10 μg of isolated total RNA of quadriceps femoris and liver were electrophoretically separated on a 1% formaldehyde agarose gel. After transfer to a Hybond N nylon membrane (Amersham, Piscataway, N.J., USA), the membranes were hybridized with a single stranded (ss) antisense human S100A1 specific DNA probe which was amplified with NFS100A1NB s (5′-cccaagctttattgcggtagt-3′; SEQ ID No. 45) and NFS100A1NB as (5′-ggctagcctatagtgagtcgtatt-3′; SEQ ID No. 46) and labelled with ³²P-dCTP in a PCR-like reaction (Fechner et al., 1999) using the luciferase antisense primer. Loading of equal amounts of RNA, was verified by rehybridization with a ss antisense β-actin specific DNA probe as previously described (Fechner, Pinkert, 2007). To detect Firefly and Renilla luciferase mRNA in 293 cotransfected cells, total RNA was isolated using Trizol (Invitrogen, Life Technologies) according to the recommendation of the supplier and 10 μg of RNA electrophoretically separated. To amplify Firefly and Renilla luciferase DNA probe, primers FFLucNB s 5′-ttcgctaagagcaccctgat-3′ (SEQ ID No. 47) and FFLucNB as 5′-ctcgtcccagtaggcaatgt-3′ (SEQ ID No. 48), RenillaNB s 5′-ccctgatcaagagcgaagag-3′ (SEQ ID No. 49) and RenillaNB as 5′-catttcatctggagcgtcct-3′ (SEQ ID No. 50) were used. Hybridized filters were exposed to Kodak Biomax MS film (Integra Biosciences, Fernwald, Germany).

### Western Blot Analysis

Proteins were extracted with general lysis buffer (20 mM Tris, pH 8.0, 10 mM NaCl, 0.5% Triton X-100, 5 mM EDTA and 3 mM MgCl₂ containing a protease inhibitor mixture (Sigma, Sigma-Aldrich). Protein concentration was determined using the BCA Protein Assay Kit (Thermo Fisher Scientific Inc.). Protein samples (5 μg of heart, 20 μg of liver and 25 μg of quadriceps femoris) were electrophorelly separated on 4-12% polyacrylamide gels (NuPAGE® Bis-Tris Gels, Invitrogen, Life Technologies) and transferred to a polyvinylidene difluoride membrane (Immun-Blot® PVDF membrane, Bio-rad Laboratories). Membranes were blocked with blocking buffer (5% milk in PBS plus 0.1% Tween-20) for 1 h at room temperature. An incubation with anti-FLAG M2 (1:1000; Agilent Technologies, Inc., Santa Clara, Calif., USA) and anti-GAPDH (1:20.000; Millipore GmbH, Schwalbach/Ts, Germany) for 1 h at room temperature followed. Membranes were washed three times with PBS plus 0.1 Tween-20 and incubated with secondary antibody conjugated to horseradish peroxidase (1:2000; Dako Denmark A/S, Glostrup, DK). Images were taken using the Biorad Molecular Imager Chemidoc XRS+ with Image Lab Software (Bio-rad Laboratories).

### ELISA

For detection of FLAG fusion proteins, anti-FLAG high sensitivity, M2 coated 96-well plates (Sigma, Sigma-Aldrich) were loaded with proteins from heart, liver and quadriceps femoris extracted with general lysis buffer in different concentrations for 1.5 h at room temperature. After washing three times with PBS plus 0.1% Tween-20, plates were incubated with anti-S100A1 (1:2000; Acris Antibodies GmbH, Herford, Germany) for 1.5 h at room temperature. Plates were washed three times with PBS plus 0.1% Tween-20 and incubated with secondary antibody (1:5000) conjugated to horseradish peroxidase (Dako Denmark NS) for 1 h at room temperature. Antibodies were diluted in PBS plus 0.1 Tween-20/0.1% BSA. Plates were washed five times with PBS plus 0.1% Tween-20. After applying of substrate (Pierce TMB ELISA substrate, Thermo Fisher Scientific Inc.), absorbance was measured using the Sunrise microplate absorbance reader for 96-well plates (Tecan Deutschland GmbH, Crailsheim, Germany).

### In Vivo Gene Transfer

All procedures involving the use and care of animals were performed according to the Guide for the Care and Use of Laboratory Animals published by the US National Institutes of Health (NIH Publication No. 85-23, revised 1996) and the German animal protection code. Approval was also granted by the local ethics review board.

1×10¹² vector genome copies/mouse of AAV9 vectors were intravenously injected into the vena jugularis of 6 week old Balb/C mice (Charles River Laboratories, Sulzfeld, Germany). After 16 days, animals were euthanized by cervical dislocation. Organs were dissected and rapidly frozen in liquid nitrogen. For histological analyses, tissue was embedded individually in tissue freezing medium (Tissue-Tek OCT Compound, Sakura Finetek Germany GmbH, Staufen, Germany) and frozen in liquid nitrogen.

### Statistical Analysis

Results are expressed as mean±standard error of mean (S.E.M.). To test for statistical significance of in vitro data, an unpaired Student's t-test was applied. Statistical significance of in vivo data was determined using the Mann-Whitney U test.

1. Isolated miR-206 target site, comprising or consisting of a nucleic acid sequence with a sequence identity of at least 80% compared to wild type miR-206 target site with SEQ ID No. 1, characterized in that the nucleic acid sequence comprises at least one nucleotide substitution at a position from nucleotide 2 to 8 and/or at least one mutation at a position from nucleotide 12 to 16 of SEQ D No. 1, wherein the nucleotide positions of SEQ ID No. 1 are numbered from the 3′- to the 5′-end.

2. Isolated miR-206 target site of claim 1, wherein the nucleic acid sequence comprises more than one nucleotide substitution, wherein each nucleotide substitution is at a position selected from nucleotide 2, 3, 4, 5, 6, 7, 8, 12, 13, 14, 15, 16 of SEQ ID No. 1, wherein the nucleotide positions of SEQ ID No. 1 are numbered from the 3′- to the 5′-end.

3. Isolated miR-206 target site of claim 1, comprising or consisting of a nucleic acid sequence with SEQ ID No. 1, wherein the nucleic acid sequence comprises one nucleotide substitution at a position selected from nucleotide 2, 3, 4, 5, 6, 7, 8, 12, 13, 14, 15, 16 of SEQ ID No. 1, wherein the nucleotide positions of SEQ ID No. 1 are numbered from the 3′- to the 5′-end.

4. Isolated miR-206 target site of claim 1, 2 or 3, wherein the mutation is a transition or a transversion.

5. Isolated miR-206 target site of one of the preceding claims, comprising or consisting of a nucleic acid sequence of SEQ ID No. 2, SEQ ID No. 3, SEQ ID No. 4, SEQ ID No. 5, SEQ ID No. 6, SEQ ID No. 7, SEQ ID No. 8, SEQ ID No. 9, SEQ ID No. 10, SEQ ID No. 11, SEQ ID No. 12, SEQ ID No. 13, SEQ ID No. 14, SEQ ID No. 15 or SEQ ID No. 22.

6. Expression cassette comprising a promoter, a nucleic acid sequence to be expressed, and at least one isolated miR-206 target site of one of claims 1 to 5.

7. Expression cassette of claim 6, wherein the nucleic acid to be expressed comprises an open reading frame encoding for a polypeptide.

8. Expression cassette of claim 6 or 7, wherein the isolated miR-206 target site of one of claims 1 to 5 is located in a 3′-untranslated region (3′-UTR) of the nucleic acid sequence to be expressed.

9. Expression cassette of one of claims 6 to 8, wherein the expression cassette comprises more than one isolated miR-206 target sites of one of claims 1 to 5.

10. Expression cassette of one of claims 6 to 9, further comprising at least one other miR target site, preferably at least one miR-122 target site.

11. Vector comprising at least one isolated miR-206 target site of one of claims 1 to 5 or at least one expression cassette of one of claims 6 to 10.

12. Vector comprising a nucleic acid sequence to be expressed operably linked to a promoter and at least one isolated miR-206 target site of one of claims 1 to 5, wherein the isolated miR-206 target site of one of claims 1 to 5 is located in a 3′-untranslated region (3′-UTR) of the nucleic acid sequence to be expressed.

13. Vector of claim 11 or 12, wherein the vector is a plasmid or a viral vector, preferably an adeno-associated viral vector.

14. Vector of one of claims 11 to 13, wherein the nucleic acid to be expressed comprises a nucleic acid sequence encoding for a polypeptide.

15. Vector of one of claims 11 to 14, further comprising at least one other miR target site, preferably at least one miR-122 target site.

16. Pharmaceutical composition comprising an isolated miR-206 target site of one of claims 1 to 5, an expression cassette of one of claims 6 to 10 or a vector of one of claims 11 to 15 and at least one pharmaceutically acceptable excipient.

17. Method of treatment of a disease comprising the step of administering a patient in need of such treatment an effective amount of a pharmaceutical composition of claim 16.

18. Method of treatment of claim 17, wherein the disease is a heart disease, preferably coronary heart disease, cardiomyopathy, cardiovascular disease, ischaemic heart disease, heart failure, hypertensive heart disease, inflammatory heart disease and/or valvular heart disease.
